SEQUENCE LISTING (11 sequences)

SEQ ID NO: 1 - protein, 639 aa, Fusarium graminearum (Gibberella zeae
ASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 2 - protein, 680 aa, Fusarium graminearum (Gibberella zeae
MKHFLSLALCFSSINAVAVTVPHKSGGTGSPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQ

SEQ ID NO: 3 - protein, 605 aa, Aspergillus niger
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGTNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAEHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 4 - protein, 354 aa, Humicola insolens
MAPKTSTFLASLTGAALVAAHGHVSHIIVNGVQYRNYDPTTDFYSGNPPTVIGWSALNQDNGFIEPNNFGTPDIICHKSAKPGGGHVTVRAGDKISIVWTPEWPESHVGPVIDYLAACNGDCETVDKTSLRFFKIDGAGYDAAAGRWAADALRANGNSWLVQIPADLKAGNYVLRHEIIALHGAANPNGAQAYPQCINIRVTGGGNNQPSGVPGTQLYKASDPGILFNPWVANPQYPVPGPALIPGAVSSIPQSRSTATATGTATRPGADTDPTGVPPVVTTTSAPAQVTTTTSSRTTSLPQITTTFATSTTPPPPAATQSKWGQCGGNGWTGPTVCAPGSSCNKLNDWYSQCI

SEQ ID NO: 5 - protein, 731 aa, Bos Taurus
MPAEDIRHPEKQPNSSKGAMWVCLQLPVFLASVTLFEVAASDTIAQAASTTTISDAVSKVKIQVNKAFLDSRTRLKTTLSSEAPTTQQLSEYFKHAKGRTRTAIRNGQVWEESLKRLRRDTTLTNVTDPSLDLTALSWEVGCGAPVPLVKCDENSPYRTITGDCNNRRSPALGAANRALARWLPAEYEDGLALPFGWTQRKTRNGFRVPLAREVSNKIVGYLDEEGVLDQNRSLLFMQWGQIVDHDLDFAPETELGSNEHSKTQCEEYCIQGDNCFPIMFPKNDPKLKTQGKCMPFFRAGFVCPTPPYQSLAREQINAVTSFLDASLVYGSEPSLASRLRNLSSPLGLMAVNQEAWDHGLAYLPFNNKKPSPCEFINTTARVPCFLAGDFRASEQILLATAHTLLLREHNRLARELKKLNPHWNGEKLYQEARKILGAFIQIITFRDYLPIVLGSEMQKWIPPYQGYNNSVDPRISNVFTFAFRFGHMEVPSTVSRLDENYQPWGPEAELPLHTLFFNTWRIIKDGGIDPLVRGLLAKKSKLMNQDKMVTSELRNKLFQPTHKIHGFDLAAINLQRCRDHGMPGYNSWRGFCGLSQPKTLKGLQTVLKNKILAKKLMDLYKTPDNIDIWIGGNAEPMVERGRVGPLLACLLGRQFQQIRDGDRFWWENPGVFTEKQRDSLQKVSFSRLICDNTHITKVPLHAFQANNYPHDFVDCSTVDKLDLSPWASREN

SEQ ID NO: 6 - protein, 639 aa, Artificial Sequence
  Synthetic
ASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 7 - protein, 639 aa, Artificial Sequence
  Synthetic
ASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDFEDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 8 - protein, 639 aa, Artificial Sequence
  Synthetic
ASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSYRNDRFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPHFEDSDATTNAHIITLGEPGTSPNTVFASNGLYFARTYHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 9 - protein, 639 aa, Artificial Sequence
  Synthetiic
ASAPIGSAIPRNNWAVTCDSAQSGNECNKAIDGNKDTFWHTFYGANGDPKPPHTYTIDMKTTQNVSGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSWRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 10 - protein, 639 aa, Artificial Sequence
  Synthetic
ASAPIGSAIPRNNWAVTCDSAQSGNECNKAIDGNKDTFWHTFYGANGDPKPPHTYTIDMKTTQNVSGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSWRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSAKTWTFLPNAKVNPMLTADKQGLYKRDNHAWLFGWKKGSVFQAGPSTAMNWYYTSGSGDVKSAGKRQSNRGVDPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKLNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 11 - protein, 638 aa, Artificial Sequence
  Synthetic
ASAPIGSAIPRNNWAVTCDSAQSGNECNKAIDGNKDTFWHTFYGANGPKPPHTYTIDMKTTQNVSGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSYRNDRFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSAKTWTFLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPHFEDSDATTNAHIITLGEPGTSPNTVFASNGLYFARTYHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

(19) United States
(12) Patent Application Publication
(10) Pub. No.: US 2023/0121990 A1
(43) Pub. Date: Apr. 20, 2023

(54) COMPOSITIONS AND METHODS FOR PRODUCTION OF GLUCOSE OXIDATION PRODUCTS

(71) Applicant: Solugen, Inc., Houston, TX (US)

(72) Inventors: Toni M. Lee, Missouri City, TX (US); Shuai Qian, Houston, TX (US); Brian F. Fisher, Houston, TX (US); Sarah Downing, Houston, TX (US); Gaurab Chakrabarti, Houston, TX (US); Sean Hunt, Houston, TX (US)

(73) Assignee: Solugen, Inc., Houston, TX (US)

(21) Appl. No.: 17/905,736

(22) PCT Filed: Mar. 6, 2021
(86) PCT No.: PCT/US2021/021261

§ 371 Date: Sep. 6, 2022

Related U.S. Application Data
(60) Provisional application No. 62/986,447, filed on Mar. 6, 2020.

(51) Int. Cl. C12P 17/06; C12P 17/04; C12N 9/04; C12N 9/08; C12N 9/02

(52) U.S. Cl. CPC C12P 17/06; C12P 17/04; C12N 9/0006; C12N 9/0065; C12N 9/0083; C12Y 101/03004; C12Y 101/03009; C12Y 111/01007; C12Y 114/99

(57) ABSTRACT

A chemoenzymatic process for the preparation of an oxidized glucose product comprising contacting D-glucose with an enzyme selected from the group consisting essentially of galactose oxidase (GAO), glucose oxidase (GOX), polysaccharide monooxygenase, catalase, animal peroxidase, periplasmic aldehyde oxidase (Pao), unspecific peroxygenase (UPO), lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), alpha-dioxygenase (aDox), dual oxidase (DuOx), prostaglandin H synthase (PGHS), cyclooxygenase (CyOx), linoleate diol synthase (LDS), variants thereof, and combinations thereof under conditions suitable for the formation of an oxidized intermediate; and contacting the oxidized intermediate with a metal catalyst to form an oxidized glucose product.

### CROSS-REFERENCE TO RELATED APPLICATIONS

This application is a 35 U.S.C. § 371 national stage application of PCT/US2021/021261, entitled “Compositions and Methods for Production of Glucose Oxidation Products,” filed Mar. 6, 2021, which claims benefit of U.S. provisional patent application Ser. No. 62/986,447 filed Mar. 6, 2020, and entitled “Compositions and Methods for Glucaric Acid Production from Glucose,” each of which is hereby incorporated herein by reference in its entirety for all purposes.

### REFERENCE TO AN ELECTRONIC SEQUENCE LISTING

The contents of the electronic sequence listing entitled “(3416-00116) Sequence Listing.xml” of size 19,376 bytes and created on Sep. 6, 2022, which is filed herewith, is incorporated herein by reference in its entirety.

### STATEMENT REGARDING FEDERALLY SPONSORED RESEARCH OR DEVELOPMENT

Not applicable.

### FIELD

The present disclosure relates to the production of high purity glucose oxidation products. More particularly, this disclosure relates to the chemoenzymatic synthesis of high purity glucaric acid and guluronic acid under mild conditions.

### BACKGROUND

The United States Department of Energy published a landmark report titled “Top Value-Added Chemicals from Biomass,” in which it highlighted a dozen molecules as the most promising framework molecules that could potentially replace commonly used petroleum-based molecular building blocks. The sugar acids glucaric acid and guluronic acid are platform chemicals in the production of some these top-value added chemicals from biomass.

Sugar acids are primarily derived from the oxidation of plant biomass-derived chemicals (e.g. glucose) and thus are accordingly considered carbon neutral, renewable chemicals. Three main classes of sugar acids exist: 1) aldonic, where the terminal aldehyde group of an aldose is oxidized to a carboxylic acid, 2) uronic, where the terminal hydroxyl group is oxidized to a carboxylic acid, and 3) aldaric, where the terminal hydroxyl and aldehyde are both oxidized to carboxylic acids to generate a diacid (e.g. glucaric acid). Guluronic acid is the uronic acid of gulose, a C-3 epimer of galactose.

Guluronic acid shares many properties with and is readily oxidized to its diacid form, glucaric acid. Glucaric acid is used primarily as an additive in detergents due to its non-toxic nature, but is also employed as a food ingredient, soap, corrosion inhibitor, de-icer, medication, and cancer treatment. The ban on the use of phosphates in detergents due to their toxic nature has increased glucaric acid demand in this segment. Although it is currently produced at lower scale relative to D-gluconic acid, the only sugar acid in high commercial production, glucaric acid is considered to have great potential as a future petrochemical replacement. Guluronic acid may be substituted for glucaric acid in any of the above applications. In addition, the L-guluronate monomer may be useful as a nonsteroidal anti-inflammatory drug.

The two main methods of generating glucaric acid for commercial use are 1) nitric acid oxidation and 2) palladium or platinum catalyst oxidation. Nitric acid oxidation generates a significant amount of hazardous nitrogen oxide (NOx) gas and is highly exothermic leading to controllability issues. A microbial method of producing high-purity glucaric acid using S. cerevisiae myo-inositol-1-phosphate synthase, mouse myo-inositol oxygenase, and P. syringae uronate dehydrogenase was developed. Microbial methods, however, can suffer from product separation issues leading to high chemical costs, limiting usage as a commodity chemical.

An ongoing need exists for novel compositions, methods and processes for the production of high purity sugar acids.

### SUMMARY

Disclosed herein is a chemoenzymatic process for the preparation of an oxidized glucose product comprising contacting D-glucose with an enzyme selected from the group consisting essentially of galactose oxidase (GAO), glucose oxidase (GOX), polysaccharide monooxygenase, catalase, animal peroxidase, periplasmic aldehyde oxidase (Pao), unspecific peroxygenase (UPO), lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), alpha-dioxygenase (aDox), dual oxidase (DuOx), prostaglandin H synthase (PGHS), cyclooxygenase (CyOx), linoleate diol synthase (LDS), variants thereof, and combinations thereof under conditions suitable for the formation of an oxidized intermediate; and contacting the oxidized intermediate with a metal catalyst to form an oxidized glucose product.

Also disclosed herein is a chemoenzymatic process for the production of glucaric acid comprising contacting glucose with a galactose oxidase having any of SEQ ID NO.:6 to SEQ ID NO.:11. under conditions suitable for formation of D-glucohexodialdose; contacting D-glucohexodialdose with a glucose oxidase having SEQ ID NO.:3 under conditions suitable for formation of L-guluronic acid-δ-2,6-lactone; and contacting L-guluronic acid-δ-2,6-lactone with a heterogeneous metal catalyst under conditions suitable for the formation of glucaric acid.

Also disclosed herein is a chemoenzymatic process for the production of D-glucono-δ-1,5-lactone comprising contacting glucose with a galactose oxidase having any of SEQ ID NO.:6 to SEQ ID NO.:11. and a glucose oxidase having SEQ ID NO.:3 under conditions suitable for the formation of D-glucono-δ-1,5-lactone.

Also disclosed herein is a chemoenzymatic process for the production of glucaric acid comprising acidifying D-glucono-δ-1,5-lactone to form L-gluconate; contacting L-gluconate with a galactose oxidase having any of SEQ ID NO.:6 to SEQ ID NO.:11. and a glucose oxidase having SEQ ID NO.:3 under conditions suitable for the formation of L-guluronate: and contacting L-guluronate with a heterogeneous metal catalyst to form glucaric acid.

Also disclosed herein is a chemoenzymatic process for the production of glucaric acid comprising contacting a polysaccharide monooxygenase having SEQ ID NO.:4. under conditions suitable for formation of saccharic acid lactone; and hydrolyzing saccharic acid lactone at a pH of greater than about 7 to form glucaric acid.

Also disclosed herein is a chemoenzymatic process for the production of glucaric acid comprising contacting glucose with an enzyme composition comprising a glucose oxidase having SEQ ID NO.:3, a peroxidase, halide ions, and a nitroxyl radical mediator, under conditions suitable for the formation to form an oxidized glucose intermediate; and contacting the oxidized glucose intermediate with a heterogeneous catalyst under conditions suitable for the formation of glucaric acid.

Also disclosed herein is a manufacturing process comprising introducing to a reactor a feedstock comprising glucose and an enzyme selected from the group consisting essentially of galactose oxidase (GAO), glucose oxidase (GOX), polysaccharide monooxygenase, catalase, animal peroxidase, periplasmic aldehyde oxidase (Pao), unspecific peroxygenase (UPO), lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), short peroxidockerin (Pxt), alpha-dioxygenase (aDox), dual oxidase (DuOx), prostaglandin H synthase, cyclooxygenase (PGHS/CyOx), linoleate diol synthase (LDS), variants thereof and combinations thereof; operating the reactor under conditions suitable for the formation of a feedstock comprising an oxidized glucose with an aldehyde moiety; transferring the feedstock comprising an oxidized glucose with an aldehyde moiety to another reactor comprising a heterogeneous metal catalyst; and operating the another reactor under conditions suitable for the oxidation of the feedstock.

### BRIEF DESCRIPTION OF DRAWINGS

For a detailed description of the aspects of the disclosed processes and systems, reference will now be made to the accompanying drawings in which:

FIG. 1A is a pH curve showing acidification following addition of GOX for the reactions from Example 1.

FIG. 1B is an HPLC-MS trace showing generation of L-guluronic acid overlayed with 200 mg/L L-guluronic acid standard trace for the samples from Example 1.

FIG. 2A is a pH curve showing acidification following addition of GOX for the reactions from Example 2.

FIG. 2B is an HPLC-MS trace showing generation of L-guluronic acid from Example 2.

FIG. 3 is a graph of the carbon balance of a two-step Parr reaction.

FIGS. 4 and 5 are plots of the glucose oxidation activity of the indicated GAO mutants.

FIG. 6 is a comparison of the activity of GAO-Mut47 and GAO-Mut107 on 0.5 and 2% glucose.

FIG. 7 is a plot of the residual glucose after a Parr reaction carried out at 11° C. for GAO Mut47.

FIG. 8A is a plot of the specific activity of machine learning mutants compared with GAO-mut47 and GAO-Mut107 controls.

FIG. 8B plots the T₅₀ of machine learning mutants compared with GAO-mut47 and GAO-Mut107 controls.

FIG. 9A depicts the glucose concentration before and after the first step of a GAO reaction.

FIG. 9B is a plot of the two-step reaction time course showing the glucose, gluconic acid and L-guluronic acid concentration.

FIGS. 9(C)-9(G) show HPLC traces with appropriate authentic standards at different M/z channel in negative mode.

FIG. 10 is a graph of the specific activity of GAO mutants on gluconate.

FIG. 11 is a plot of the reaction time course showing the concentration of dextrose, gluconic acid, and L-guluronic acid as measured via HPLC-MS.

FIG. 12 is a schematic view of a manufacturing process of the type disclosed herein.

### DETAILED DESCRIPTION

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997) can be applied, as long as that definition does not conflict with any other disclosure or definition applied herein, or render indefinite or non-enabled any claim to which that definition is applied.

Groups of elements of the periodic table are indicated using the numbering scheme indicated in the version of the periodic table of elements published in Chemical and Engineering News, 63(5), 27, 1985. In some instances, a group of elements can be indicated using a common name assigned to the group; for example alkali metals for Group 1 elements, alkaline earth metals for Group 2 elements, transition metals for Group 3-12 elements, and halogens for Group 17 elements, among others.

Regarding claim transitional terms or phrases, the transitional term “comprising,” which is synonymous with “including,” “containing,” “having,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. The phrase “consisting essentially of” occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials that do not significantly alter the composition or method to which the term is applied. While compositions and methods are described in terms of “comprising” various components or steps, the compositions and methods can also “consist essentially of” or “consist of” the various components or steps.

As noted above, the two main methods of generating glucaric acid for commercial use are 1) nitric acid oxidation and 2) palladium or platinum catalyst oxidation. Nitric acid oxidation generates a significant amount of hazardous nitrogen oxide (NOx) gas and is highly exothermic leading to controllability issues. A microbial method of producing high-purity glucaric acid using S. cerevisiae myo-inositol-1-phosphate synthase, mouse myo-inositol oxygenase, and P. syringae uronate dehydrogenase was developed. Microbial methods, however, can suffer from product separation issues leading to high chemical costs, limiting usage as a commodity chemical. An ongoing need exists for novel compositions, methods and processes for the production of high purity sugar acids.

While catalysts can oxidize aldehyde moieties to carboxylic acids in the presence of primary and secondary alcohols, it remains extremely challenging to selectively oxidize a primary alcohol when other secondary alcohols are present. State-of-the-art catalytic systems generally result in an array of side products including ketoses, ketoacids, and over-oxidation degradation products.

To solve this problem, a primary alcohol oxidation is sepeated into two reactors. In the first reactor, a cell-free enzymatic system can be used that can selectively oxidizes primary alcohols to aldehydes in the presence of secondary alcohols and other functional groups. In the second reactor, a heterogeneous metal catalyst can selectively oxidize the aldehyde moieties to carboxylic acids while preserving the secondary alcohols and carbon-carbon bond arrangements. As described in various embodiments, this process technology can be used with a variety of feedstocks such as a glucose feedstock, which contains one aldehyde at C1, one primary alcohol at C6, and 4 secondary alcohols. Oxidation of the C6 primary alcohol can be performed enzymatically in Reactor 1 to obtain a glucohexodialdose (glucodialdose) intermediate. Although a dialdehyde with four secondary alcohols is anticipated to be unstable, this intermediate can be oxidized to glucaric acid in Reactor 2 using a heterogeneous metal catalyst with high activity and selectivity. The combination of the enzymatic and heterogeneous catalytic systems results in an efficient manufacturing process for the production of products such as glucaric acid, guluronic acid, or both.

Disclosed herein are chemoenzymatic methods for the production of glucaric acid, guluronic acid, or both. In an aspect, glucaric acid, guluronic acid or both are produced from glucose. The chemoenzymatic methods disclosed herein may comprise contacting glucose with one or more biocatalysts, one or chemical catalysts, and one or more metal catalysts. The chemoenzymatic methods disclosed herein may result in intermediates that can be further processed and provide useful value-added chemicals.

In an aspect, a method of the present disclosure is depicted in Scheme I below. Referring to Scheme 1, as shown in Pathway A, glucose isomerizes between α-D-glucose and β-D-glucose. Glucose may be contacted with a galactose oxidase (GAO) variant under conditions suitable for oxidation of the C6 alcohol to an aldehyde generating D-glucohexodialdose. D-glucohexodialdose (also known as D-glucodialdose) is then contacted with a glucose oxidase (GOX) under conditions suitable for oxidation of the C1 alcohol to produce L-guluronic acid-δ-2,6-lactone. L-guluronic acid-δ-2,6-lactone, which is in equilibrium with L-guluronic acid, may be harvested directly or further reacted with a heterogeneous metal catalyst (HMC) under conditions suitable for the formation of glucaric acid.

In an alternative aspect, depicted as Pathway B of Scheme 1, a GAO variant and GOX are simultaneously contacted with glucose under conditions suitable for the production D-glucono-δ-1,5-lactone. In one or more aspects, D-glucono-δ-1,5-lactone is further processed and isolated as a product. In the alternative, D-glucono-δ-1,5-lactone is acidified to form gluconate which is contacted with a GAO under conditions suitable for the formation of L-guluronate. Acidification may be carried using any suitable acidizing agent (e.g., HCl). L-guluronate may be contacted with an HMC under conditions suitable for the formation of glucaric acid.

In an alternative aspect, depicted in Scheme II, a GAO variant is contacted with glucose under conditions suitable for oxidation of the C6 alcohol of glucose to an aldehyde generating the dialdehyde D-glucohexodialdose. D-glucohexodialdose is contacted with an HMC under conditions suitable for the formation of glucaric acid.

In one or more aspects, a method of producing glucaric acid comprises contacting, a polysaccharide monooxygenase (PMO) under conditions suitable for the oxidation of both the C1 and C6 alcohols of glucose to form saccharic acid lactone. This is depicted in Scheme III. The lactone is easily hydrolyzed under alkaline conditions, greater than about pH 7 to form glucaric acid. Notably saccharic acid lactone will also slowly self-hydrolyze to form the free acid under relevant reaction conditions.

In one or more aspects, PMO may be combined with a GOX to oxidize the C1 alcohol of glucose. Because PMO is also suspected of oxidizing the C4 alcohol to a ketone when provided hydrogen peroxide, catalase can be added to limit availability of this oxidizing agent, thereby suppressing the undesirable C4 keto pathway. Products from this process may also be passed over a HMC to oxidize any unreacted sugars to the diacid.

In one or more aspects, glucose is contacted with an enzymatic oxidizing composition (EOC) comprising a GOX, an animal peroxidase (XPO), halide ions, and a nitroxyl radical mediator (NRM). Herein, a “halide” has its usual meaning; therefore, examples of halides include fluoride, chloride, bromide, and iodide. Referring to Scheme IV, glucose is contacted with an NRM under conditions suitable for the formation of D-glucohexodialdose. D-glucohexodialdose is then contacted with GOX under conditions suitable for the formation of D-guluronic acid-δ-1,5-lactone, which can be converted to glucaric acid in the presence of a HMC. In the alternative, glucose is contacted first with a GOX under conditions suitable for the formation of D-glucono-δ-1,5-lactone. NRMs may be included in the reaction to promote formation of the D-glucoronic acid-δ-1,5-lactone from D-glucono-δ-1,5-lactone and its subsequent oxidation to glucaric acid using an HMC. A schematic of peroxidase-driven NRM recycling is presented in Scheme V where R₁₋₅ represent the same or different akyl groups. R₆ represents a ketone or alcohol.

Referring to Scheme VI, glucose may be contacted GAO under conditions suitable for the formation of D-glucohexodialdose. D-glucohexodialdose may optionally be contacted with GAO to generate D-guluronic acid. D-glucohexodialdose or D-guluronic acid may then be contacted with a periplasmic aldehyde oxidase (Pao) or unspecific peroxygenase (UPO) to form glucaric acid.

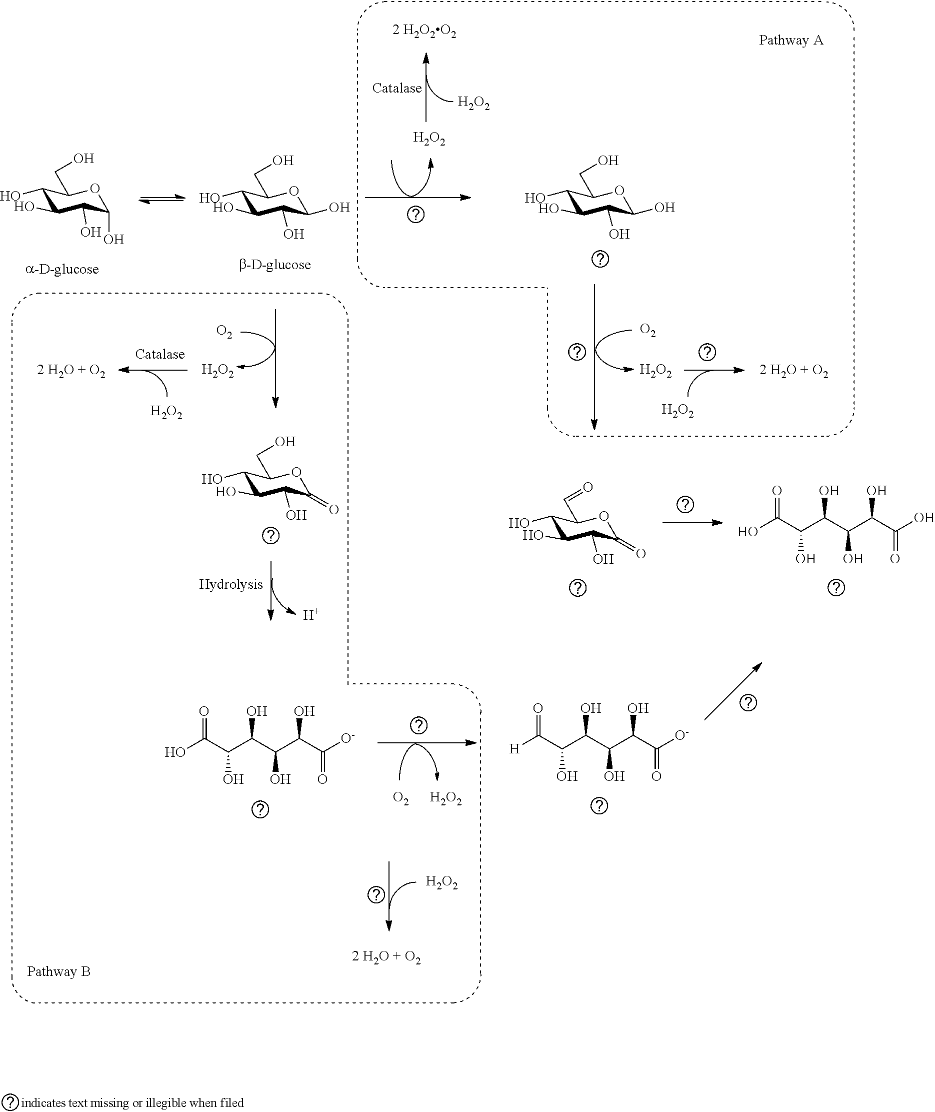

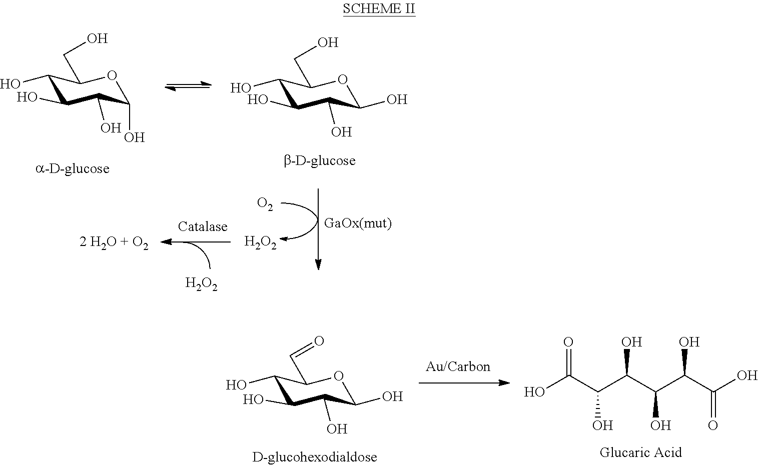

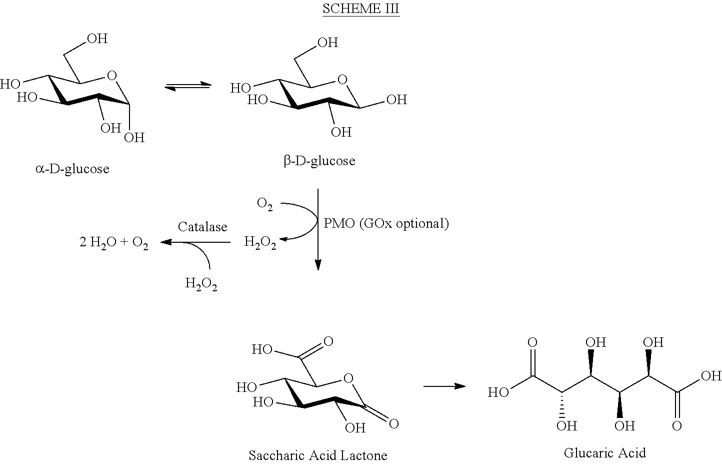

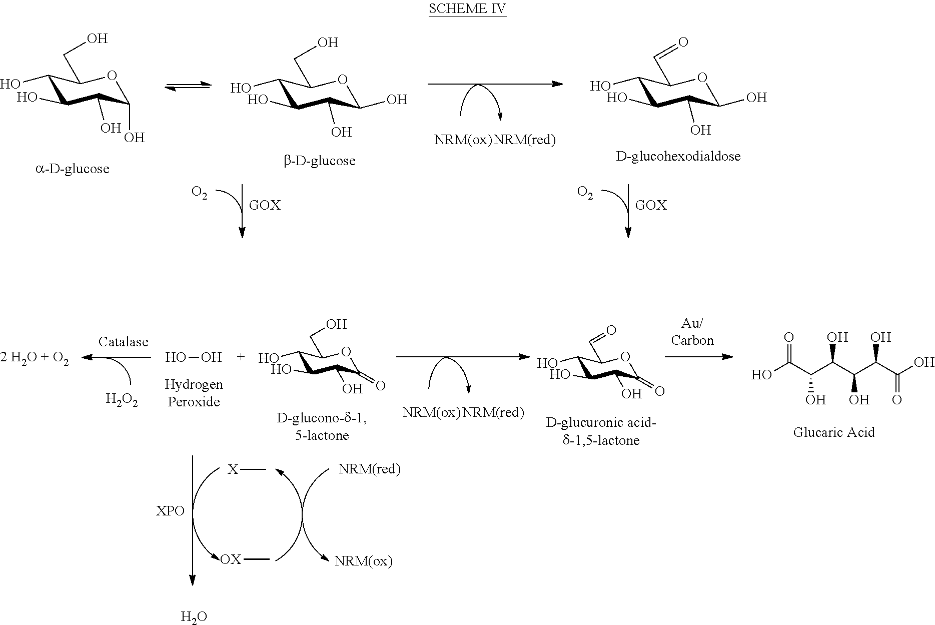

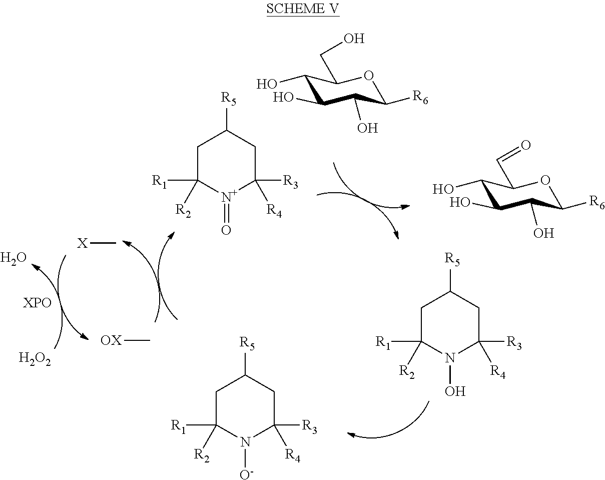

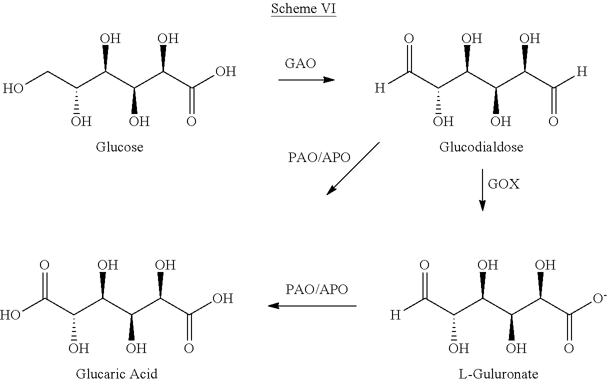 [SMILES: O=C(O)[C@H](O)[C@@H](O)[C@H](O)[C@H](O)CO ; O=C[C@@H](O)[C@@H](O)[C@H](O)[C@@H](O)C=O ; O=C(O)[C@@H](O)[C@@H](O)[C@H](O)[C@@H](O)C(=O)O ; O=C[C@@H](O)[C@@H](O)[C@H](O)[C@@H](O)C(=O)[O-]]

In an aspect, a biocatalyst suitable for use in the present disclosure is selected from the group consisting essentially of galactose oxidase (GAO), glucose oxidase (GOX), polysaccharide monooxygenase (PMO), catalase, animal peroxidase, periplasmic aldehyde oxidase, unspecific peroxygenase, lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), alpha-dioxygenase (aDox), dual oxidase (DuOx), prostaglandin H synthase (PGHS), cyclooxygenase (CyOx), linoleate diol synthase (LDS), variants thereof and combinations thereof. Herein, the term “biocatalyst” and “enzyme” are used interchangeably.

In an aspect, the biocatalyst is a member of the copper radical oxidase family. For example, and without limitation, a copper radical oxidase suitable for use in the present disclosure is galactose oxidase (GAO, EC 1.1. 3.9). GAO is one of the most extensively studied alcohol oxidases with respect to both mechanistic investigations and practical applications. Other members in the copper radical oxidase family may be suitably employed in the present disclosure.

GAO is secreted by some fungal species, particularly Fusarium graminearum (also known as Gibberella zeae), to aid in the degradation of extracellular carbohydrate food sources through catalyzing the oxidation of primary alcohols to aldehydes while generating hydrogen peroxide. The native function of GAO is the oxidation of D-galactose at the C6 position to generate D-galacto-hexodialdose. A small molecule (potassium ferricyanide) or auxiliary enzyme (i.e., horseradish peroxidase or HRP) is typically included to facilitate GAO activity. Typically, HRP is added to the reaction at a tenth of the weight percent (wt. %) of GAO. Catalase is also added to decompose hydrogen peroxide. Although the GAO is promiscuous, the native form is unable to bind glucose due to steric clashes with F464 and F194 in the active site and the equatorial C4 hydroxyl group on glucose. Efforts to engineer GAO to accept D-glucose as a substrate to form the C6 aldehyde have resulted in improved activity as shown in Table 1. The M-RQW variant (R330K, Q406T, W290F) shows a specific activity of 1.6 U mg⁻¹. Another variant, the Des3-2 (Q326E, Y329K, R330K) showed four times higher activity on glucose than the native enzyme. In addition, the mutation C383S was found to improve catalytic efficiency up to three times by reducing the K_{M} of the enzyme on non-native substrates guar gum and methylgalactose through improved binding of the catalytic copper ion. Table 1 provides a listing of some GAO mutants that may be useful in the methods of the present disclosure.

TABLE 1

| Enzyme | | |
|---|---|---|
| Name | Mutations | Benefits |
| M-RQW | R330K Q406T W290F | 1.6 U/mg on glucose |
| Des3-2 | Q326E Y329K R330K | 4× activity on glucose vs WT |
| NA | C383S | Reduces K_{M} through improved |
| | | copper binding |
| M1 | S10P M70V P136 G195E | Improves E. coli expression and |
| | V494A N535D | solubility |
| N6M1 | A2A (GCC→GCA) S3S | Silent N-term mutations for |
| | (TCA→TCT) I5I | enhanced E. coli expression |
| | (ATC→ATT) | |
| NA | F194T C383E N245W/R | Improved specific activity of up |
| | | to 3 U/mg with N245R and |
| | | C383E in N6M1 background |
| NA | W290F R330K Q406T | 1.6 μM/min conversion on |
| | Y405F Q406E | glucose |

In an aspect, a GAO suitable for use in the present disclosure may have any of SEQ ID NO.:1, SEQ ID NO.2:, or SEQ ID NO.:6 to SEQ ID NO.:11.

In an aspect, the biocatalyst is a GOX. Glucose oxidase (EC number 1.1.3.4, herein “GOX”) is a soluble, homodimeric, secreted flavoprotein that oxidizes β-D-glucose (a natural isomerization product in equilibrium with α-D-glucose) to D-glucono-δ-1,5-lactone while reducing molecular oxygen to form hydrogen peroxide. GOX is commercially available for many uses including the determination of free glucose in sera or blood plasma for diagnostics, as a monitoring agent in fermentation processes, for controlling glucose in vegetal raw material and food products, as an additive in baked goods or egg products, or as an oxygen removal agent in packaged foods. In an aspect, an exemplary GOX suitable for use in the present disclosure has SEQ ID NO: 3.

In an aspect, the biocatalyst is a polysaccharide monooxygenase (E.C. 1.14.99.56, PMO). PMOs, also known as lytic PMOs (LPMOs), enhance the depolymerization of recalcitrant polysaccharides by hydrolytic enzymes and are found in the majority of cellulolytic fungi and actinomycete bacteria. For more than a decade, PMOs were incorrectly annotated as family 61 glycoside hydrolases (GH61s) or family 33 carbohydrate-binding modules (CBM33s). PMOs have an unusual surface-exposed active site with a tightly bound Cu(II) ion that catalyzes the regioselective hydroxylation of crystalline cellulose, leading to glycosidic bond cleavage. In an aspect, an exemplary PMO suitable for use in the present disclosure has SEQ ID NO: 4.

In an aspect, the biocatalyst is a peroxidase. Peroxidases (EC 1.11.1.x) belong to a large family of isoenzymes present in almost all living organisms. These are generally heme containing enzymes ranging in molecular weight from about 35 kilodaltons (kD) to about 100 kD. Mammalian peroxidases are much larger proteins (576-738 amino acids) than the plant counterparts. Peroxidases exist as monomers, dimmers or tetramers and their gene locations also vary among different chromosomes. For example, glutathione peroxidase 4 (GPx4) is a monomer, eosinophil peroxidase (EPO) exists as a dimer, while glutathione peroxidase 1 (GPx1) is a homotetramer.

In an aspect, the biocatalyst is a glutathione peroxidase. Glutathione peroxidases (GPx) are heme thiol peroxidases, comprising a family of eight isoenzymes (GPx1-8) with diverse functions besides catalyzing the reduction of H₂O₂ or organic hydroperoxides to water or alcohols. GPx1 is the most abundant among GPx family proteins as it is found in erythrocytes and other tissues. It protects these cells from harmful effects of H₂O₂ produced by coupled oxidation of different hydrogen donors with oxyhemoglobin.

In an aspect, the biocatalyst is a thyroid peroxidase. Thyroid peroxidase also called thyroperoxidase (TPO) is mainly expressed in thyroid organs. It is a large transmembrane glycoprotein with covalently linked haem, present in cells on the apical membrane.

In an aspect, the biocatalyst is a lactoperoxidase. Lactoperoxidase (LPO) is found in a wide range of mammalian and human tissues, glands and their secretions. LPO contributes to the non-immune host defense system, plays an important role against pathogenic microorganisms and has a protective role in respiratory tract. In an aspect, an exemplary LPO suitable for use in the present disclosure has SEQ ID NO: 5.

In an aspect, the biocatalyst is a salivary/oral peroxidase (SPO). SPO is a component of the first line of defense system present in saliva, Oral peroxidases OPO are composed of salivary peroxidase (80%) and MPO (20%). Salivary peroxidase also forms an oral antioxidant system.

In an aspect, the biocatalyst is an eosinophil peroxidase (EPO). Eosinophil granulocytes or eosinophils are type of white blood cells actively involved in immune system against multicellular parasites and other infections. Eosinophil granules contain a good quantity of eosinophil peroxidase (EPO) (40%) which performs a vast majority of functions during different diseased states. EPO is actively involved in Cl—, Br—, I— and SCN—oxidation.

In an aspect, the biocatalyst is a myeloperoxidase (MPO). MPO is packed inside the cytoplasmic azurophilic granules of neutrophils and is involved in unspecific immune defense system responsible for microbicidal activity. MPO catalyzes lipid peroxidation via tyrosyl radical formation and this leads to generation of other products which cause lipoprotein oxidation.

In an aspect, the biocatalyst is an ovoperoxidase (OPO). OPO is one of several oocyte-specific proteins that are stored within sea urchin cortical granules, released during the cortical reaction, and incorporated into the newly formed fertilization envelope. Ovoperoxidase plays a particularly important role in this process, crosslinking the envelope into a hardened matrix that is insensitive to biochemical and mechanical challenges and thus providing a permanent block to polyspermy.

In an aspect, the biocatalyst is a vanadium haloperoxidase (VHPO). In the environment, VHPOs are likely to play a key role in the production of biogenic organohalogens. These enzymes contain vanadate as a prosthetic group, and catalyze, in the presence of hydrogen peroxide, the oxidation of halide ions (Cl—, Br— or I—). They are classified according to the most electronegative halide that they can oxidize.

In an aspect, the biocatalyst is a peroxidasin. Peroxidasin is a novel protein combining peroxidase and extracellular matrix motifs. Cultured cells secrete peroxidasin; it occurs in larvae and adults. Each 1512 residue chain of the three-armed, disulfide-linked homotrimer combines a peroxidase domain with six leucine-rich regions, four Ig loops, a thrombospondin/procollagen homology and an amphipathic alpha-helix. The peroxidase domain is homologous with human myeloperoxidase and eosinophil peroxidase. This heme protein catalyzes H₂O₂-driven radioiodinations, oxidations and formation of dityrosine.

In an aspect, the biocatalyst is a α-dioxygenases (α-DOX). α-DOXs oxygenate fatty acids into 2(R)-hydroperoxides. Despite the low level of sequence identity, α-DOX share common catalytic features with cyclooxygenases (COX), including the use of a tyrosyl radical during catalysis.

In an aspect, the biocatalyst is an invertebrate peroxynectin. A 76-kDa protein that mediated attachment and spreading of the crayfish blood cells was purified from blood cells of the crayfish Pacifastacus leniusculu. The deduced protein sequence was significantly similar to one family of peroxidases, e.g., myeloperoxidase. Peroxinectin is the first cell adhesion molecule cloned from invertebrate blood and the first protein from any organism that combines being a cell adhesion ligand and a peroxidase.

In an aspect, the biocatalyst is a Prostaglandin E synthase (PGES). PGES converts cyclooxygenase (COX)-derived prostaglandin (PG)H2 to PGE2, occurs in multiple forms with distinct enzymatic properties, modes of expression, cellular and subcellular localizations and intracellular functions. Cytosolic PGES (cPGES) is a cytosolic protein that is constitutively expressed in a wide variety of cells and tissues and is associated with heat shock protein 90 (Hsp90).

In an aspect, the biocatalyst is a linoleate diol synthase (EC 1.13.11.44, LDS). LDS is an enzyme that utilizes the two substrates of this enzyme, linoleate and O₂ to generate (9Z,12Z)-(7S,8S)-dihydroxyoctadeca-9,12-dienoate. LDS belongs to the family of oxidoreductases, specifically those acting on single donors with O₂ as oxidant and incorporation of two atoms of oxygen into the substrate (oxygenases). The oxygen incorporated need not be derived from O₂. The systematic name of this enzyme class is linoleate:oxygen 7S,8S-oxidoreductase. This enzyme is also called linoleate (8R)-dioxygenase.

In an aspect, the biocatalyst is a periplasmic aldehyde oxidoreductase (Pao). PaoABC from Escherichia coli is a molybdenum enzyme involved in detoxification of aldehydes in the cell. It is an example of an αβγ heterotrimeric enzyme of the xanthine oxidase family of enzymes which does not dimerize via its molybdenum cofactor binding domain.

In an aspect, the biocatalyst is an unspecific peroxygenase (UPO). Unspecific peroxygenase (EC 1.11.2.1, aromatic peroxygenase, mushroom peroxygenase, haloperoxidase-peroxygenase, Agrocybe aegerita peroxidase) is an enzyme with systematic name substrate:hydrogen peroxide oxidoreductase (RH-hydroxylating or -epoxidising). Unspecific peroxygenase is a heme-thiolate protein comparable to Cytochrome P450 in the ability to catalyze a variety of P450 reactions (hence “unspecific”), but forms a unique, solely fungal, protein family of extracellular enzymes.

In some aspects, the biocatalyst is selected from the group consisting of E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1. B2 chloride peroxidase; E.C. 1.11.1. B6 iodide peroxidase (vanadium-containing); E.C. 1.11.1. B7 bromide peroxidase or combinations thereof.

In an aspect, a biocatlaays of the present disclosure is a catalase (E.C. 1.11.1.61). CAT is a tetrameric, heme-containing, antioxidant enzyme present in all aerobic organisms. Catalase catalyzes the decomposition of H₂O₂ into water and oxygen.

Any of the biocatalysts disclosed herein may be a wild type enzyme, a functional fragment thereof, or a functional variant thereof. As used herein, “fragment” is meant to include any amino acid sequence shorter than the full-length enzyme (e.g., GAO), but where the fragment maintains a catalytic activity sufficient to meet some user or process goal. Fragments may include a single contiguous sequence identical to a portion of the enzyme sequence. Alternatively, the fragment may have or include several different shorter segments where each segment is identical in amino acid sequence to a different portion of the amino acid sequence of the enzyme but linked via amino acids differing in sequence from the enzyme. Herein, a “functional variant” of the enzyme refers to a polypeptide that has at one or more positions of an amino acid insertion, deletion, or substitution, either conservative or non-conservative, and wherein each of these types of changes may occur alone, or in combination with one or more of the others, one or more times in a given sequence but retains catalytic activity.

In the alternative or in combination with the aforementioned mutations, a biocatalyst may be mutated to improve the catalytic activity. Mutations may be carried out in order to enhance the protein or a homolog activity, increase the protein stability in the presence of products and/or hydrogen peroxide, and increase protein yield.

Herein, reference is made to “sources” of biocatalysts or enzymes. It is to be understood this refers to the biomolecule as expressed by the named organism. It is contemplated the enzyme may be obtained from the organism or a version of said enzyme (wildtype or recombinant) provided as a suitable construct to an appropriate expression system.

In an aspect, any enzyme of the type disclosed herein may be cloned into an appropriate expression vector and used to transform cells of an expression system such as E. coli, Saccharomyces sp., Pichia sp., Aspergillus sp., Trichoderma sp., or Myceliophthora sp. A “vector” is a replicon, such as plasmid, phage, viral construct, or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs), and the like into which one or more gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of an enzyme disclosed herein is provided as a recombinant sequence in a vector where the sequence is operatively linked to one or more control or regulatory sequences. “Operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and refer to polynucleotide sequences, which are necessary to affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

In an aspect, a biocatalyst of the type disclosed herein may further include one or more purified cofactors. Herein, a “cofactor” refers to non-protein chemical compound that modulates the biological activity of the enzyme. Many enzymes require cofactors to function properly. Nonlimiting examples of purified enzyme cofactors suitable for use in the present disclosure include thiamine pyrophosphate, NAD+, NADP+, pyridoxal phosphate, methyl cobalamin, cobalamine, biotin, Coenzyme A, tetrahydrofolic acid, menaquinone, ascorbic acid, flavin mononucleotide, flavin adenine dinucleotide, metals (e.g., copper), and Coenzyme F420. Such cofactors may be included in reactions disclosed herein and/or be added at various points during a reaction. In some aspects, cofactors included with the biocatalyst may be readily regenerated with oxygen and/or may remain stable throughout the lifetime of the enzyme(s).

In one or more aspects, any biocatalyst disclosed herein is present in an amount sufficient to provide some user and/or process desired catalytic activity. In such aspects, any of the biocatalysts disclosed herein may be present in an amount ranging from about 0.0001 wt. % to about 1 wt. %, alternatively from about 0.0005 wt. % to about 0.1 wt. % or alternatively from about 0.001 wt. % to about 0.01 wt. % based on the total weight of the reaction mixture.

Nitroxyl radical mediators (NRM) are a class of N-oxyl compounds represent a versatile class of organic radical reagents with unique properties and reactivity. The diverse chemistry of these compounds has enabled the use of N-oxyl species in applications ranging from use as spin labels in electron spin resonance (ESR) studies, antioxidants in biological studies, charge carriers for energy storage, mediators in polymerization reactions, and catalysts in chemical and electrochemical oxidation reactions. The two most prominent classes of N-oxyl compounds are aminoxyl and imidoxyl species, of which the two most widely used members are 2,2,6,6-tetramethylpiperidine N-oxyl (TEMPO) and phthalimide N-oxyl (PINO), respectively TEMPO is stable under ambient conditions, whereas PINO is generated via oxidation of the stable precursor, N-hydroxyphthalimide (NHPI).

In one or more aspects of the present disclosure, a final oxidation step is carried out to convert an intermediate to glucaric acid or guluronic acid. In an aspect of the present disclosure, the oxidation can be carried out using a metal catalyst, alternatively a supported metal catalyst. In an aspect, the metal catalyst comprises a supported metal catalyst such as a heterogeneous metal catalyst or a homogenous metal catalyst (HMC). In an aspect, the support comprises carbon, silica, alumina, titania (TiO₂), zirconia (ZrO₂), a zeolite, or any combination thereof, which contains less than about 1.0 weight percent (wt. %), alternatively less than about 0.1 wt. % or alternatively less than about 0.01 wt. % SiO₂ binders based on the total weight of the support.

Suitable support materials are predominantly mesoporous or macroporous, and substantially free from micropores. For example, the support may comprise less than about 20% micropores. In an aspect, the support of the HMC is a porous nanoparticle support. As used herein, the term “micropore” refers to pores with a diameter of <2 nm, as measured by nitrogen adsorption and mercury porosimetry methods and as defined by IUPAC. As used herein, the term “mesopore” refers to pores with a diameter of from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In an aspect, the HMC support comprises a mesoporous carbon extrudate having a mean pore diameter ranging from about 10 nm to about 100 nm, and a surface area greater than about 20 m² g⁻¹ but less than about 300 m² g⁻¹. Supports suitable for use in the present disclosure may have any suitable shape. For example, the support may be shaped into 0.8-3.0 mm trilobes, quadralobes, or pellet extrudates. Such shaped supports enable the used of fixed trickle bed reactors to perform the final oxidation step under continuous flow.

In an aspect, the HMC comprises metals of main group IV, V, VI, alternatively the metal is from subgroup I, IV, V, VII, alternatively the HMC comprises gold, Au. In one or more aspects, the metal comprises a Group 8 metal (e.g., Re, Os, Ir, Pt, Ru, Rh, Pd, Ag), a 3d transition metal, an early transition metal, or a combination thereof. In an alternative aspect, a dehydration catalyst comprises hafnium, tantalum, zinc, or a combination thereof on a support such as a zeolite or a p-zeolite. In an aspect, a metal catalyst suitable for use in the present disclosure comprises a metal oxide, zirconia doped with alkaline-earth elements, rare earth orthophosphate catalyst, ruthenium, or a combination thereof.

The HMC may be prepared using any suitable methodology. For example, the HMC may be prepared using gas phase reduction of the support (e.g., carbon) impregnated with metal salts in hydrogen at temperatures ranging from greater than about 200° C. to about 600° C. In an alternative aspect, the HMC may be prepared using liquid phase reduction of the support impregnated with metal salts immersed in an aqueous oxygenate (e.g., formate, gluconate, citrate, ethylene glycol, etc.) solution at temperature between about 0° C. and about 100° C. Alternatively, the impregnated support can be loaded into the hydrogenation reactor in a non-reduced form and reduced on stream by the reactants of the process during startup. Liquid Phase Reduction (LPR) is a synthetic method to obtain a core-shell dispersion of the active metallurgy over a surface annulus of the extrudate.

In an aspect, materials of the type disclosed herein are prepared via incipient wetness or bulk adsorption of a metal precursor salt solution onto the extrudate support followed by either Gas Phase Reduction (GPR) at temperatures between 100° C. and 500° C. under an H₂/N₂ atmosphere or followed by Liquid Phase Reduction (LPR) using an alkaline aqueous solution.

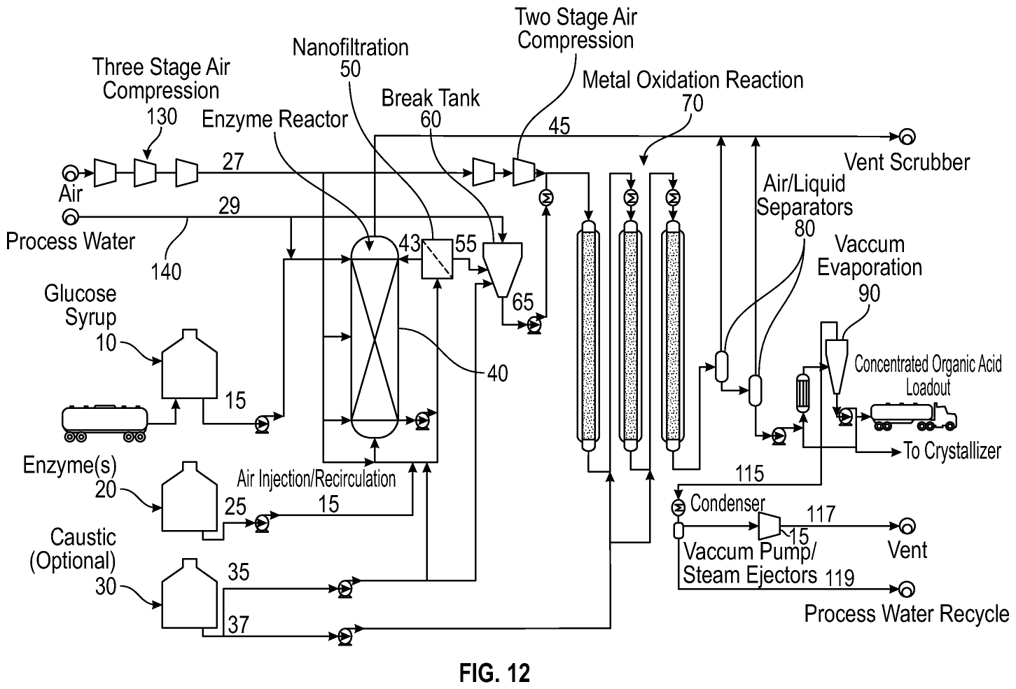

In an aspect, chemoenzymatic processes of the type disclosed herein may be carried out in any suitable manufacturing system 200. An aspect of a suitable manufacturing system 200 is depicted in FIG. 12. Referring to FIG. 12, reactants such as glucose and enzyme may be introduced to an enzyme reactor 40 from containers 10 and 20 via lines 15 and 25, respectively. In one or more aspects of the methodologies disclosed, the pH of the reaction may be adjusted to an alkaline range (i.e., greater than about 7). In such instances, a caustic agent, such as a suitable base (e.g., NaOH, KOH), may be introduced to any reactor or other downprocess vessel from a tank containing the caustic 30 via lines 35 or 37. In an aspect, any of the components of the manufacturing system 200 may have air and/or process water introduced from a process water source 130 via line 29 or a compressor 140 via line 27. Materials exiting enzyme reactor 40 may be conveyed through a nanofiltration unit 50 via conduit 43 to a break tank 60 before being further processed by conveyance via conduit 65 to a series of reactors comprising a HMC of the type disclosed herein, 70. Material exiting the series of reactors comprising a HMC 70 may be further processed such as through air liquid separators 80, or a vacuum evaporator 90.

Although the above drawing is at PFD level of detail, not all process interconnections are shown such as spillbacks, block and bleeds, recycle lines, control valves, cooling/heating elements, pumps, intermediate tankage, antifoam, etc.

In an aspect, the methods disclosed herein result in the preparation of high purity oxidation products of glucose. For example, the glucose oxidation products (e.g., glucaric acid, guluronic acid) may have a purity of greater than about 80%, alternatively greater than about 85%, alternatively greater than about 95%, alternatively from about 80% to about 99%, alternatively from about 85% to about 99%, or alternatively from about 90% to about 99%.

### ADDITIONAL DISCLOSURE

The following enumerated aspects of the present disclosures are provided as non-limiting examples.

A first aspect which is a chemoenzymatic process for the preparation of an oxidized glucose product comprising contacting D-glucose with an enzyme selected from the group consisting essentially of galactose oxidase (GAO), glucose oxidase (GOX), polysaccharide monooxygenase, catalase, animal peroxidase, periplasmic aldehyde oxidase (Pao), unspecific peroxygenase (UPO), lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), alpha-dioxygenase (aDox), dual oxidase (DuOx), prostaglandin H synthase (PGHS), cyclooxygenase (CyOx), linoleate diol synthase (LDS), variants thereof, and combinations thereof under conditions suitable for the formation of an oxidized intermediate; and contacting the oxidized intermediate with a metal catalyst to form an oxidized glucose product.

A second aspect which is the chemoenzymatic process of the first aspect, wherein the galactose oxidase has SEQ ID NO.:1.

A third aspect which is the chemoenzymatic process of any of the first through second aspects, wherein the galactose oxidase has SEQ ID NO.:2.

A fourth aspect which is the chemoenzymatic process of any of the first through third aspects, wherein the galactose oxidase has any of SEQ ID NO.:6 to SEQ ID NO.:11.

A fifth aspect which is the chemoenzymatic process of any of the first through fourth aspects, wherein the glucose oxidase has SEQ ID NO.:3.

A sixth aspect which is the chemoenzymatic process of any of the first through fifth aspects, wherein the peroxidase is a lactoperoxidase.

A seventh aspect which is the chemoenzymatic process of the sixth aspect, wherein the lactoperoxidase has SEQ ID NO.:5.

An eighth aspect which is the chemoenzymatic process of any of the first through seventh aspects, wherein the polysaccharide monooxygenase has SEQ ID NO.:4.

A ninth aspect which is the chemoenzymatic process of any of the first through eighth aspects, carried out at a temperature of less than about 100° C.

A tenth aspect which is the chemoenzymatic process of any of the first through ninth aspects, wherein the oxidized glucose product has a purity of greater than about 80%.

An eleventh aspect which is the chemoenzymatic process of any of the first through tenth aspects, wherein the oxidized glucose product comprises guluronic acid.

A twelfth aspect which is the chemoenzymatic process of any of the first through tenth aspects, wherein the oxidized glucose product comprises glucaric acid.

A thirteenth aspect which is a chemoenzymatic process of any of the first through twelfth aspects, wherein the metal catalyst comprises a support comprising carbon, silica, alumina, titania (TiO₂), zirconia (ZrO₂), zeolite, or any combination thereof.

A fourteenth aspect which is the chemoenzymatic process of any of the first through thirteenth aspects, wherein the metal catalyst is homogeneous.

A fifteenth aspect which is the chemoenzymatic process of any of the first through fourteenth aspects, wherein the metal catalyst is heterogeneous.

A sixteenth aspect which is a chemoenzymatic process for the production of glucaric acid comprising contacting glucose with a galactose oxidase having any of SEQ ID NO.:6 to SEQ ID NO.:11. under conditions suitable for formation of D-glucohexodialdose; contacting D-glucohexodialdose with a glucose oxidase having SEQ ID NO.:3 under conditions suitable for formation of L-guluronic acid-δ-2,6-lactone; and contacting L-guluronic acid-δ-2,6-lactone with a heterogeneous metal catalyst under conditions suitable for the formation of glucaric acid.

A seventeenth aspect which is a chemoenzymatic process for the production of D-glucono-δ-1,5-lactone comprising contacting glucose with a galactose oxidase having any of SEQ ID NO.:6 to SEQ ID NO.:11. and a glucose oxidase having SEQ ID NO.:3 under conditions suitable for the formation of D-glucono-δ-1,5-lactone.

An eighteenth aspect which is a chemoenzymatic process for the production of glucaric acid comprising acidifying D-glucono-δ-1,5-lactone to form L-gluconate; contacting L-gluconate with a galactose oxidase having any of SEQ ID NO.:6 to SEQ ID NO.:11. and a glucose oxidase having SEQ ID NO.:3 under conditions suitable for the formation of L-guluronate: and contacting L-guluronate with a heterogeneous metal catalyst to form glucaric acid.

A nineteenth aspect which is a chemoenzymatic process for the production of glucaric acid comprising contacting a polysaccharide monooxygenase having SEQ ID NO.:4. under conditions suitable for formation of saccharic acid lactone; and

hydrolyzing saccharic acid lactone at a pH of greater than about 7 to form glucaric acid.

A twentieth aspect which is a chemoenzymatic process for the production of glucaric acid comprising contacting glucose with an enzyme composition comprising a glucose oxidase having SEQ ID NO.:3, a peroxidase, halide ions, and a nitroxyl radical mediator, under conditions suitable for the formation to form an oxidized glucose intermediate; and contacting the oxidized glucose intermediate with a heterogeneous catalyst under conditions suitable for the formation of glucaric acid.

A twenty-first aspect which is a chemoenzymatic process of the twentieth aspect, wherein the nitroxyl radical mediator comprises 2,2,6,6-tetramethylpiperidine N-oxyl (TEMPO) phthalimide N-oxyl or a combination thereof.

A twenty-second aspect which is a manufacturing process comprising introducing to a reactor a feedstock comprising glucose and an enzyme selected from the group consisting essentially of galactose oxidase (GAO), glucose oxidase (GOX), polysaccharide monooxygenase, catalase, animal peroxidase, periplasmic aldehyde oxidase (Pao), unspecific peroxygenase (UPO), lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), short peroxidockerin (Pxt), alpha-dioxygenase (aDox), dual oxidase (DuOx), prostaglandin H synthase, cyclooxygenase (PGHS/CyOx), linoleate diol synthase (LDS), variants thereof and combinations thereof; operating the reactor under conditions suitable for the formation of a feedstock comprising an oxidized glucose with an aldehyde moiety; transferring the feedstock comprising an oxidized glucose with an aldehyde moiety to another reactor comprising a heterogeneous metal catalyst; and operating the another reactor under conditions suitable for the oxidation of the feedstock.

A twenty-third aspect which is a two-step manufacturing process wherein the first reactor, an enzymatic system is used to oxidize a feedstock containing at least 1 primary alcohol to an aldehyde, and where in the second reactor, a heterogeneous catalyst is used to oxidize the corresponding aldehydes of the feedstock intermediate to carboxylic acids.

A twenty-fourth aspect which is the manufacturing process of the twenty-third aspect where the primary alcohol is the C6 alcohol group of a glucose feedstock.

A twenty-fifth aspect which is the manufacturing process of any of the twenty-third through twenty-fourth aspects where the first reactor comprises an engineered galactose oxidase, catalase, and a peroxidase system.

A twenty-sixth aspect which is the manufacturing process of any of the twenty-third through twenty-fifth aspects where the product of the first reactor is glucodialdose.

A twenty-seventh aspect which is the manufacturing process of any of the twenty-third through twenty-sixth aspects where the first reactor is a sparged, pressurized bubble column.

A twenty-eighth aspect which is the manufacturing process of any of the twenty-third through twenty-seventh aspects where the first reactor is a fermenter.

A twenty-ninth aspect which is the manufacturing process of any of the twenty-third through twenty-eighth aspects where the first reactor is an air-lift bubble column.

A thirtieth aspect which is the manufacturing process of any of the twenty-third through twenty-ninth aspects where the first reactor operates at a pH between 1 and 12, a temperature between 0 and 100 Celsius, and a pressure between 1 and 100 bar.

A thirty-first aspect which is the manufacturing process of any of the twenty-third through thirtieth aspects where the first reactor operates without the addition of stoichiometric cations

A thirty-second aspect which is the manufacturing process of any of the twenty-third through thirty-first aspects where the second reactor is a trickle bed reactor.

A thirty-third aspect which is the manufacturing process of any of the twenty-third through thirty-second aspects where the second reactor is a continuous stirred tank reactor.

A thirty-fourth aspect which is the manufacturing process of any of the twenty-third through thirty-third aspects where the second reactor is a slurry plug flow reactor.

A thirty-fifth aspect which is the manufacturing process of any of the twenty-third through thirty-fourth aspects where the second reactor operates at a temperature between 50 and 200 Celsius, a pressure between 10 and 200 bar.

A thirty-sixth aspect which is the manufacturing process of any of the twenty-third through thirty-fifth aspects where the second reactor operates without the addition of stoichiometric cations.

A thirty-seventh aspect which is the manufacturing process of any of the twenty-third through thirty-sixth aspects where the heterogeneous catalyst comprises supported gold nanoparticles.

A thirty-eighth aspect which is the manufacturing process of any of the twenty-third through thirty-seventh aspects where the heterogeneous catalyst support is carbon, titania, or zirconia.

A thirty-ninth aspect which is the manufacturing process of any of the twenty-third through thirty-eighth aspects where the heterogeneous catalyst comprises a multimetallic gold alloy.

A fortieth aspect which is the manufacturing process of any of the twenty-third through thirty-ninth aspects where the alloying metal is platinum.

A forty-first aspect which is the manufacturing process of any of the twenty-third through fortieth aspects where the products of the second reactor are uronic and aldaric acids.

A forty-second aspect which is the manufacturing process of any of the twenty-third through forty-first aspects where the products of the second reactor are guluronic, glucuruonic, and glucaric acid.

A forty-third aspect which is the manufacturing process of any of the twenty-third through forty-second aspects where the products of the second reactor are sent to a separating system that enables the separation and front-end recycling of feedstocks and intermediate molecules.

A forty-fourth aspect which is the manufacturing process of any of the twenty-third through forty-third aspects wherein the separating system is Sequential Simulated Moving Bed Chromatography (SSMB).

A forty-fifth aspect which is the manufacturing process of any of the twenty-third through forty-third aspects where the final product is de-watered via evaporation.

A forty-sixth aspect which is the manufacturing process of any of the twenty-third through forty-fifth aspects where the final product is purified via crystallization and drying.

A forty-seventh aspect which is a two-step manufacturing process where in the first reactor, an enzymatic system is used to oxidize a feedstock containing at least 1 primary alcohol to a carboxylic acid, and where in the second reactor, a heterogeneous catalyst is used to oxidize any remaining aldehyde moieties in the feedstock to the corresponding carboxylic acid moieties.

A forty-eighth aspect which is the manufacturing process of the forty-seventh aspect where the primary alcohol is the C6 alcohol group of a glucose feedstock.

A forty-ninth aspect which is the manufacturing process of any of the forty-seventh through forty-eighth aspects where the first reactor comprises an engineered galactose oxidase, glucose oxidase, catalase, and a peroxidase system.

A fiftieth aspect which is the manufacturing process of any of the forty-seventh through forty-ninth aspects where the product of the first reactor is the guluronate anion and corresponding lactones.

A fifty-first aspect which is the manufacturing process of any of the forty-seventh through fiftieth aspects where the product of the first reactor is the glucuronate anion and corresponding lactones.

A fifty-second aspect which is the manufacturing process of any of the forty-seventh through fifty-first aspects where the product of the first reactor is the glucarate anion and corresponding lactones.

A fifty-third aspect which is the manufacturing process of any of the forty-seventh through fifty-second aspects where the first reactor is a sparged, pressurized bubble column.

A fifty-fourth aspect which is the manufacturing process of any of the forty-seventh through fifty-third aspects where the first reactor is a fermenter.

A fifty-fifth aspect which is the manufacturing process of any of the forty-seventh through fifty-fourth aspects where the first reactor is an air-lift bubble column.

A fifty-sixth aspect which is the manufacturing process of any of the forty-seventh through fifty-fifth aspects where the first reactor operates at a pH between 1 and 12, a temperature between 0 and 100 Celsius, and a pressure between 1 and 100 bar.

A fifty-seventh aspect which is the manufacturing process of any of the forty-seventh through fifty-sixth aspects where the first reactor operates with the addition of stoichiometric alkali metal or alkali earth metal cations.

A fifty-eighth aspect which is the manufacturing process of any of the forty-seventh through fifty-seventh aspects where the second reactor is a trickle bed reactor.

A fifty-ninth aspect which is the manufacturing process of any of the forty-seventh through fifty-eighth aspects where the second reactor is a continuous stirred tank reactor.

A sixtieth aspect which is the manufacturing process of any of the forty-seventh through fifty-ninth aspects where the second reactor is a slurry plug flow reactor.

A sixty-first aspect which is the manufacturing process of any of the forty-seventh through sixtieth aspects where the second reactor operates at a temperature between 50 and 200 Celsius, a pressure between 10 and 200 bar.

A sixty-second aspect which is the manufacturing process of any of the forty-seventh through sixty-first aspects where the second reactor operates without the addition of stoichiometric cations.

A sixty-third aspect which is the manufacturing process of any of the forty-seventh through sixty-second aspects where the heterogeneous catalyst comprises supported gold nanoparticles.

A sixty-fourth aspect which is the manufacturing process of any of the forty-seventh through sixty-third aspects where the heterogeneous catalyst support is carbon, titania, or zirconia.

A sixty-fifth aspect which is the manufacturing process of any of the forty-seventh through sixty-fourth aspects where the heterogeneous catalyst comprises a multimetallic gold alloy.

A sixty-sixth aspect which is the manufacturing process of any of the forty-seventh through sixty-fifth aspects where the alloying metal is platinum.

A sixty-seventh aspect which is the manufacturing process of any of the forty-seventh through sixty-sixth aspects where the products of the second reactor are uronic and aldaric acids.

A sixty-eighth aspect which is the manufacturing process of any of the forty-seventh through sixty-seventh aspects where the products of the second reactor are guluronic, glucuruonic, and glucaric acid.

A sixty-ninth aspect which is the manufacturing process of any of the forty-seventh through sixty-eighth aspects where the products of the second reactor are sent to a separating system that enables the separation and front-end recycling of feedstocks and intermediate molecules.

A seventieth aspect which is the manufacturing process of any of the forty-seventh through sixty-ninth aspects wherein the separating system is Sequential Simulated Moving Bed Chromatography (SSMB).

A seventy-first aspect which is the manufacturing process of any of the forty-seventh through seventieth aspects where the final product is de-watered via evaporation.

A seventy-second aspect which is the manufacturing process of any of the forty-seventh through seventy -first aspects where the final product is purified via crystallization and drying.

### EXAMPLES

The presently disclosed subject matter having been generally described, the following examples are given as particular aspects of the subject matter and to demonstrate the practice and advantages thereof. It is understood that the examples are given by way of illustration and are not intended to limit the specification or the claims in any manner.

### Example 1

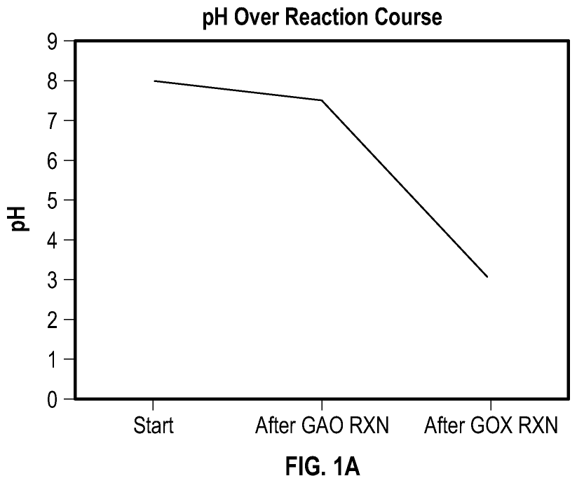

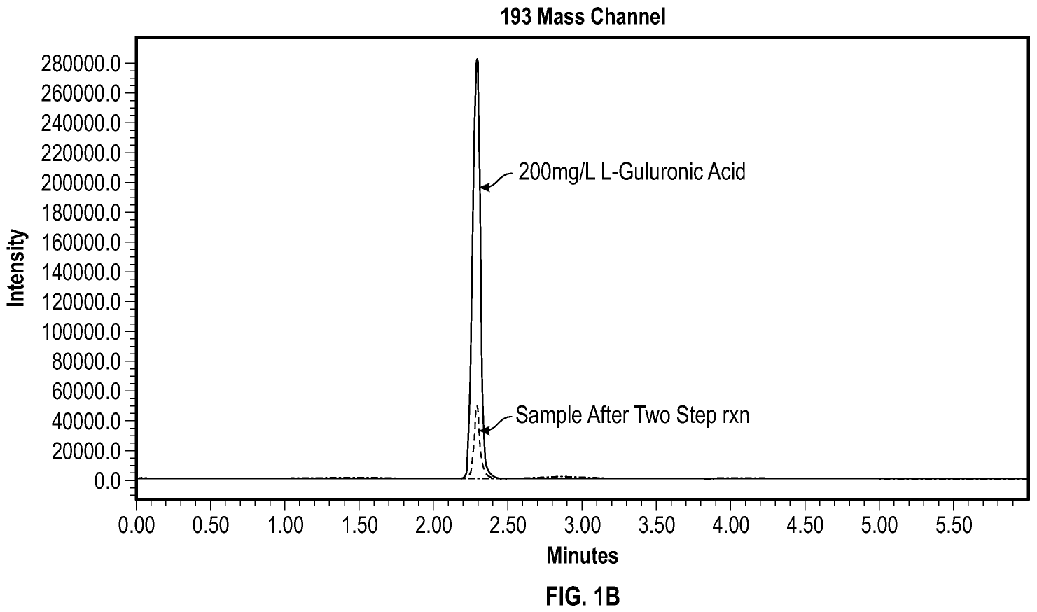

L-guluronic acid production was demonstrated at benchtop scale using GAO-mut1 and GOX enzymes added to a Parr bomb vessel pressurized to 100 psi with oxygen. In an initial experiment, a solution of 50 mM sodium phosphate buffer, pH 8, containing 10% w/v glucose, 0.02% w/v GAO-mut1, and 0.001% w/v catalase was prepared at a 50 mL volume and added to the inner chamber of the vessel. The stirred reaction proceeded at 20° C. for 20 hours. The GAO Mut-1 enzyme was then removed by filtering through a 30 kD molecular weight cutoff (MWCO) centrifugal device. In the second reaction phase, GOX and catalase were both added to a concentration of 0.001% w/v. The reaction was again allowed to proceed with stirring at 20° C. for 20 hours. It was noted that during the reaction, the pH was found to decline from 8 to 3, presumably from the generation of the acid species gluconic and L-guluronic acid after the addition of GOX and the results are shown in FIG. 1A. HPLC-MS analysis of the final reaction mix, FIG. 1B, demonstrated production of around 0.2-0.3% L-guluronic acid (2-3% molar yield), 2% gluconate, and an unspecified quantity of glucodialdose. The estimate of yield was made using a comparison of the high pressure liquid chromatography-mass spectrometry (HPLC-MS) trace showing generation of L-guluronic acid overlayed with 200 mg/L L-guluronic acid standard trace.

### Example 2

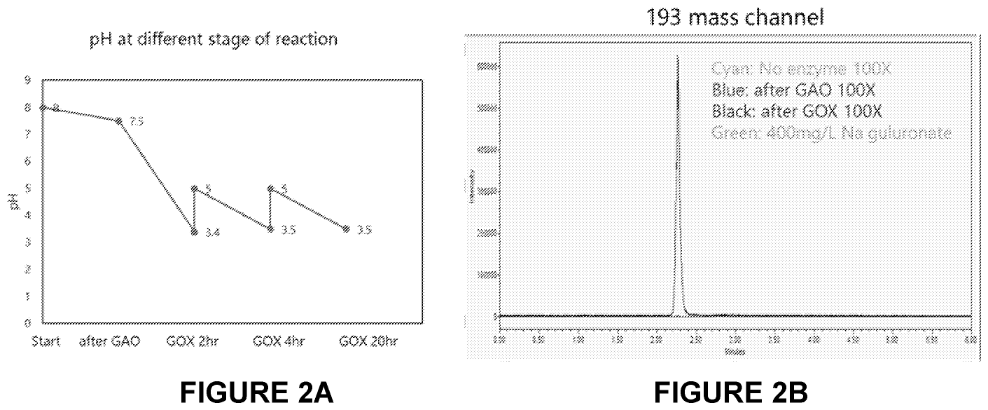

To improve yield, a second experiment was performed in which pH was controlled during the acid-generating second step. First, a solution of 50 mM sodium phosphate buffer, pH 8, 15% w/v glucose, 0.02% w/v GAO-mut1, and 0.001% w/v catalase was added to the Parr bomb at a volume of 50 mL and stirred at a temperature of 20° C. for 20 hours to generate glucodialdose. In the second step, 0.001% w/v GOX and an additional 0.001% w/v catalase was added and allowed to proceed at the same conditions for an additional 20 hours. The reaction was stopped, the pH adjusted to 5 at 2 and 4 hours, then repressurized and allowed to react until 20 hours elapsed. The results are shown in FIG. 2A. Liquid chromatography-mass spectrometry (LC-MS) analysis of the product, FIG. 2B, demonstrated a higher concentration (approximately 5% w/v or 31% molar yield) of L-guluronate was generated compared to that observed in Example 1, FIG. 1B. Referring to FIG. 1B, HPLC-MS traces showing generation of L-guluronic acid after addition of GOX overlayed with 400 mg/L L-guluronic acid standard trace. No compounds in the 193-mass channel in negative ion mode (i.e., L-guluronic acid) were detected in the no enzyme control or after addition of only GAO.

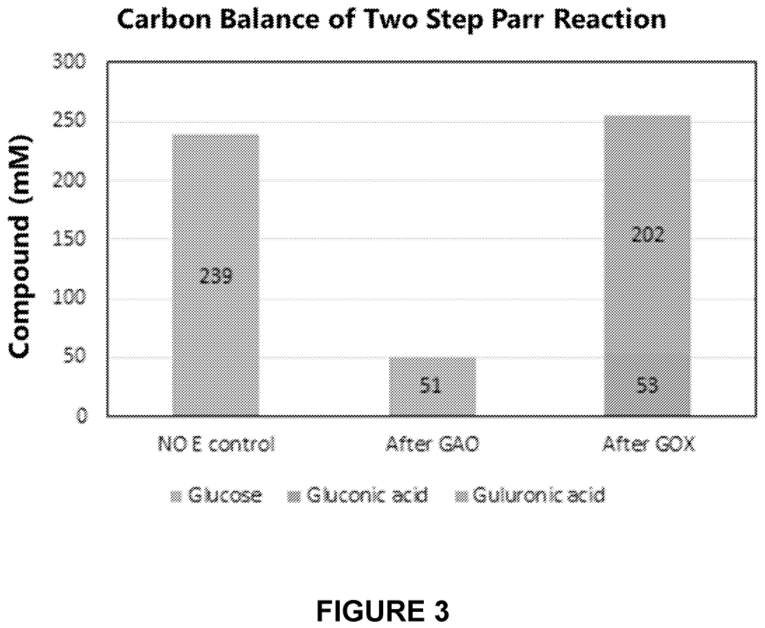

A third run was performed to further increase yield by adding a higher concentration of GAO-mut1 and adjusting pH to 6 in the second catalysis step. First, a solution of 50 mM sodium phosphate buffer, pH 8, approximately 4% w/v glucose, 0.1% w/v GAO-mut1, and 0.001% w/v catalase was added to the Parr bomb at a volume of 50 mL and stirred at a temperature of 20° C. for 20 hours to generate glucodialdose. In the second step, 0.001% w/v GOX and an additional 0.001% w/v catalase was added and allowed to proceed at the same conditions for another 20 hours. The reaction was periodically paused and the pH adjusted to 6. Following the reaction with GAO-Mut1, the concentration of glucose dropped from the initial loading of 4.3% w/v to 0.9% w/v. After addition of GOX and reaction for 20 hours, a mixture of 1.0% w/v gluconic and 3.6% w/v L-guluronic acid (85% molar yield). The carbon balance of this reaction with GAO-Mut1 and GOX was determined and the concentration in mM of glucose, gluconic acid and L-guluronic acid are shown in FIG. 3 after 20 hours of reaction with GAO, and after 20 hours or reaction with GOX and pH adjustment.

### Example 3

A GAO mutant capable of converting glucose to glucodialdose was engineered. Following directed evolution and rational enzyme engineering, the improved GAO mutant exhibits a specific activity of 35 U mg⁻¹ on glucose. Directed evolution of thirty sites within 10 Å of the catalytic copper was performed on a parent sequence containing the following added mutations: 1) R330, Q406T, W290F discovered by 2) C383S, and 3) Y405F and Q406E. Other mutations described in Table 2 were found to have neutral or deleterious effects on glucodialdose-generating activity. We named the new combination sequence GAO-Mut1. The full sequence of the expressed construct is given as SEQ ID No. 1, which contains a “MGHHHHHHSSGHIEGRHM” N-terminal his-tag and linker for expression and purification in E. coli.

Selected positions in GAO-Mut1 were mutated via the Quikchange method to all 20 amino acids using primers containing NNS codons. The constructs were then screened in the following manner: Colonies were picked and used to inoculate one well each in a 96-well deepwell plate charged with lysogeny broth (LB). The grown clones were then used to inoculate autoinduction media in a separate 96-well deepwell plate for protein expression. Harvested cells were lysed with Bacterial Protein Extraction Reagent (B-PER) and the lysate was then screened for oxidase activity using a colorimetric ABTS assay which detects hydrogen peroxide.

In short, lysate was assayed for activity with and without exposure to heat. To assay activity in the absence of a heat challenge, lysate was diluted 50 times. A volume of 5 μL of the diluted lysate was combined with ABTS assay solution (final concentration of 2% w/v glucose, 0.0125 mg/ml horseradish peroxidase, 50 mM sodium phosphate buffer at pH 8, 0.05% ABTS) to a final volume of 200 μL and the change in absorbance at 405 nm was monitored until the reaction was complete. To assay residual activity after a heat challenge, 50 μL lysate was incubated for ten minutes at 50° C. and 20 μL of the heat-treated lysate was added to the ABTS solution before monitoring change in absorbance at 405 nm. Specific activity was calculated from the formulas below using the linear portion of the curve to measure ΔA405/min and taking the extinction coefficient of ABTS at 405 nm as 36.8 mM⁻¹cm⁻¹.

$$Units {mg}^{- 1} = \frac{Δ {A}_{405} {min}^{- 1}}{36.8 \times ( pathlength in cm ) \times ( mg enzyme ) / ( ml reaction mixture )} \\ Units {ml}^{- 1} = \frac{Δ {A}_{405} {min}^{- 1}}{36.8 \times ( pathlength in cm ) \times ( mg enzyme ) / ( ml reaction mixture )}$$

Mutant lysates exhibiting a ΔA405/min greater than GAO-Mut1 were chosen for further characterization. Following identification of the mutation by DNA sequencing, hits were expressed, purified, and assayed for specific activity and thermostability as assessed by the temperature at which one half maximal activity was observed (T₅₀). Mutants were purified from 5 ml culture with auto-induction medium in a 24 well plate. Harvested cells were lysed with B-PER and the lysate was spun down with 15,000 relative centrifugal force (rcf) for 30 min at 4° C. The lysate supernatant was used for protein purification with HisPur™ Ni-NTA Spin Plates. The eluted protein sample was diluted with 100 mM potassium phosphate buffer pH 7.5 with 0.5 mM CuSO₄, and specific activity was measured using the ABTS assay. T50 was measured by heating protein in the absence of substrate, cooling, and then measuring residual activity using the ABTS assay. Heating was accomplished by diluting the protein to a concentration of 2.5 mg/L in a volume of 100 mM phosphate buffer at pH 7.5, aliquoting 50 μL into a row of a 96-well PCR plate, and incubating over a temperature gradient sufficient to capture maximal and minimal enzyme performance for ten minutes. Promptly after heating, the mixture was cooled on ice and the ΔA405/m in of 20 μL of enzyme solution in 200 μL of final volume of ABTS solution was measured as described above.

Hits were purified, tested for activity and T50, and recombined to generate a final best mutant from the directed evolution step. Promising point mutants that could beneficially be combined in the Mut1 background included A193R D404H F441Y A172V are listed in Table 4. These mutations were combined into a single combination mutant named GAO-Mut47 which exhibited a specific activity of 27.3 U mg⁻¹ and a T50 of 56.8° C. Table 2 provides a list of point mutations carried out and their characteristics.

TABLE 2

| | | | | K_{cat} | Kₘ |
|---|---|---|---|---|---|
| Name | Mutations | U/mg | T50° C. | S⁻¹ | mM |
| M-RQW-S | | 1.1 | 56.8 | 31.4 | 2168.3 |
| GAO-mut1 | Y405F Q406E | 14.0 | 51.8 | 30.2 | 93.1 |
| GAO-mut6 | Y405F Q406E S383C | 6.1 | 41.5 | 36.6 | 412.0 |
| GAO-mut7 | Y405F Q406E F441Y | 16.9 | 53.6 | 27.3 | 42.7 |
| GAO-mut8 | Y405F Q406E D404H | 15.0 | 53.7 | 30.7 | 83.9 |
| GAO-mut9 | Y405F Q406E G461A | 13.4 | 53.2 | 27.8 | 83.7 |
| GAO-mut10 | Y405F Q406E I462R | 12.1 | 53.2 | 31.6 | 130.7 |
| GAO-mut11 | Y405F Q406E A172V | 21.2 | 48.5 | 39.5 | 72.6 |
| GAO-mut12 | Y405F Q406E A193R | 15.4 | 56.3 | 28.0 | 64.8 |
| GAO-mut13 | Y405F Q406E A193T | 14.6 | 53.8 | 30.4 | 75.5 |
| GAO-mut14 | Y405F Q406E D404H F441Y | 18.8 | 55.0 | 26.5 | 29.7 |
| GAO-mut15 | Y405F Q406E G461A I462R | 12.2 | 54.0 | 24.8 | 79.1 |
| GAO-mut17 | Y405F Q406E D404H F441Y G461A | 18.2 | 55.3 | 23.6 | 25.5 |
| | I462R | | | | |
| GAO-mut18 | Y405F Q406E A193T D404H F441Y | 18.1 | 56.6 | 24.1 | 28.0 |
| | G461A I462R | | | | |
| GAO-mut19 | Y405F Q406E A193T D404H F441Y | 13.3 | 46.3 | 24.5 | 70.8 |
| | G461A I462R S383C | | | | |
| GAO-mut20 | Y405F Q406E A193T D404H F441Y | 21.4 | 37.9 | 35.6 | 58.2 |
| | G461A I462R S383C A172V | | | | |
| GAO-mut21 | Y405F Q406E F441Y G461A I462R | 18.3 | 53.8 | 24.2 | 29.6 |
| GAO-mut22 | Y405F Q406E A193T D404H F441Y | 23.6 | 51.5 | 29.5 | 26.4 |
| | G461A I462R A172V | | | | |
| GAO-mut23 | Y405F Q406E A193R D404H F441Y | 21.1 | 57.5 | 27.2 | 26.8 |
| | G461A I462R A172V | | | | |
| GAO-mut47 | Y405F Q406E A193R D404H F441Y | 27.3 | 56.8 | 35.0 | 25.2 |
| | A172V | | | | |
| GAO-mut58 | Y405F Q406E D404H F441Y A172V | 27.1 | 52.9 | 35.4 | 26.6 |

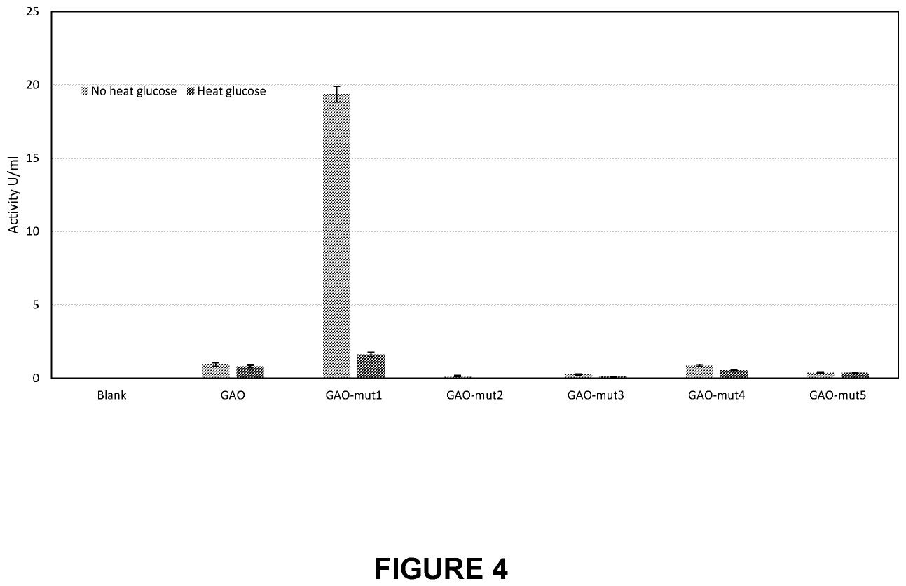

The results of assaying the activity of the indicated GAO using glucose as a substrate is presented in FIG. 4.

### Example 4

Rational engineering of GAO to further accept a glucose substrate and identify stabilizing mutations was accomplished with a combination of computational methods based on structural and multiple sequence alignment data (MSA). It was identified that GAO-M-RQW-S could accept both glucose and gluconate as substrate, the results are displayed in FIG. 2. Rational design was performed on the GAO-M-RQW-S sequence rather than GAO-Mut1. Structural methods employed included applying FoldX55 (40 predicted mutations) and PROSS56 (80 mutations) to a modified form of the Protein Database (PDB) structure 2WQ8 to contain the GAO-M-RQW-S mutations. MSA-based predictions were applied to a 185-member MSA. This MSA was generated from an initial set of 1000 sequences curated with JALVIEW to remove sequences with 98% redundancy and retain only sequences experimentally verified as carbohydrate oxidases. 30 mutations identified in designing a GAO for synthesizing an intermediate of the HIV drug Islatravir were also added to the panel.

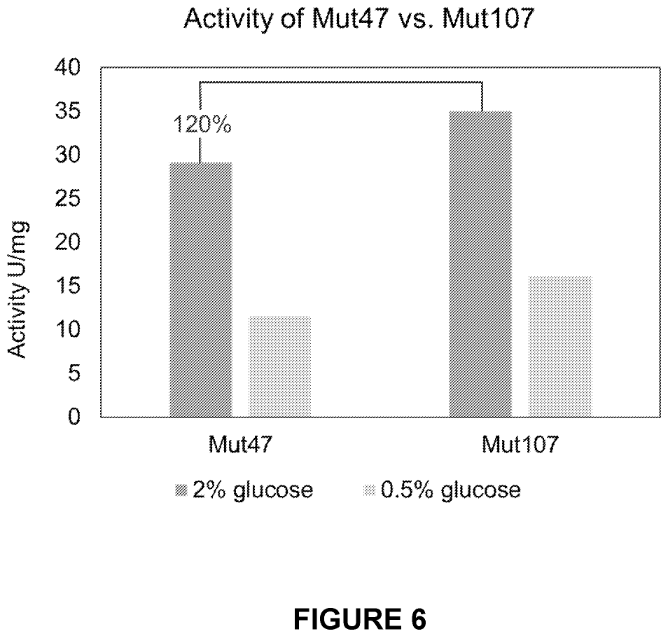

In total, 202-point mutants were screened using the same methods described above for screening the directed evolution clones. Thirty-nine hits were identified from an initial screen and sixteen were reidentified from a second round of screening. Upon generation of combo mutants in the best combination mutant from the directed evolution step (GAO-Mut47), the mutations N66S, S306A, S311F, and Q486L were identified as complementary and beneficial while N28I, Y189W, S331R, A378D, and R459Q were deemed detrimental in this background. The results are summarized in Table 3. The final GAO-Mut107 construct containing the Mut47 mutations and N66S, S306A, S311F, and Q486L exhibits a specific activity of 34.96 U mg⁻¹ on 2% glucose and a T50 of 60.56° C. as shown in FIG. 6. Additional mutations identified from machine learning algorithms were later incorporated to generate GAO-mut142 and GAO-mut164. A comparison of the activity of GAO Mut47 and Mut107 was made and the results are presented in FIG. 6.

TABLE 3

| | | New | | Fold | |
|---|---|---|---|---|---|
| Clone | Mutations from Mut47 | Mutations | U/mg | Improvement | T50° C. |
| Mut47 | | | 31.11 | 1.00 | 57.64 |
| GAO-mut68 | N28I | N28I | 30.84 | 0.99 | 56.76 |
| GAO-mut69 | N28I N66S | N66S | 33.68 | 1.08 | 59.00 |
| GAO-mut70 | N28I N66S Y189W | Y189W | 31.80 | 1.02 | 59.91 |
| GAO-mut71 | N28I N66S Y189W S306A | S306A | 32.66 | 1.05 | 59.48 |
| GAO-mut72 | N28I N66S Y189W S306A | S311F | 33.87 | 1.09 | 60.81 |
| | S311F | | | | |
| GAO-mut73 | N28I N66S Y189W S306A | S331R | 27.56 | 0.89 | 59.87 |
| | S311F S331R | | | | |
| GAO-mut74 | N28I N66S Y189W S306A | A378D | 25.57 | 0.82 | 58.94 |
| | S311F S331R A378D | | | | |
| GAO-mut75 | N28I N66S Y189W S306A | R459Q | 23.51 | 0.76 | 58.49 |
| | S311F S331R A378D R459Q | | | | |
| GAO-mut76 | N28I N66S Y189W S306A | V477D | 19.22 | 0.62 | 59.17 |
| | S311F S331R A378D R459Q | | | | |
| | V477D | | | | |
| GAO-mut77 | N28I N66S Y189W S306A | Q486L | 24.57 | 0.79 | 59.88 |
| | S311F S331R A378D R459Q | | | | |
| | V477D Q486L | | | | |
| GAO-mut107^{a} | N66S S306A S311F Q486L | Removed | 34.96 | 1.20 | 60.56 |
| | | N28I, | | | |
| | | Y189W, | | | |
| | | S331R, | | | |
| | | A378D, | | | |
| | | R459Q, | | | |
| | | and V477D | | | |
| GAO-mut142^{b} | N66S S306A S311F Q486L | H40C | 37.53 | 1.29 | 58.76 |
| | H40C | | | | |
| GAO-mut164^{b} | N66S S306A S311F Q486L | L71C | 38.22 | 1.32 | 52.97 |
| | H40C L71C | | | | |

Bolded mutations are beneficial in a Mut47 background A193R D404H F441Y A172V
^{a}Data collected in a separate experiment from other data. Fold improvement is calculated compared to an internal Mut47 control.
^{b}Data collected in a separate experiment from other data. Fold improvement is calculated compared to an internal Mut47 control.

### Example 5

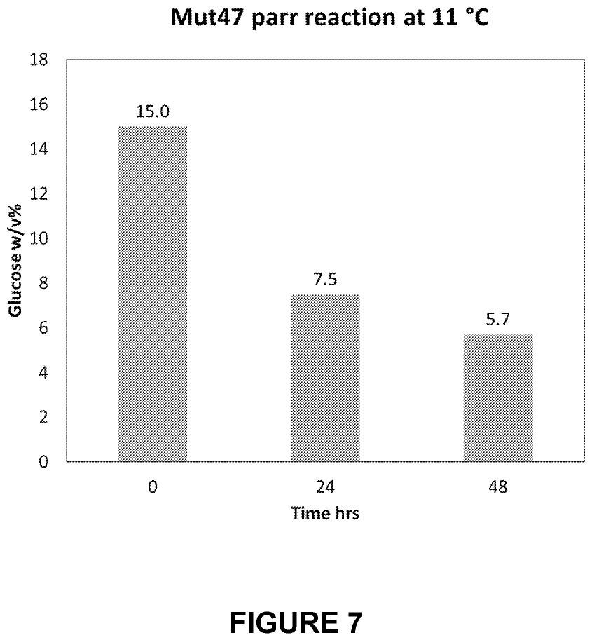

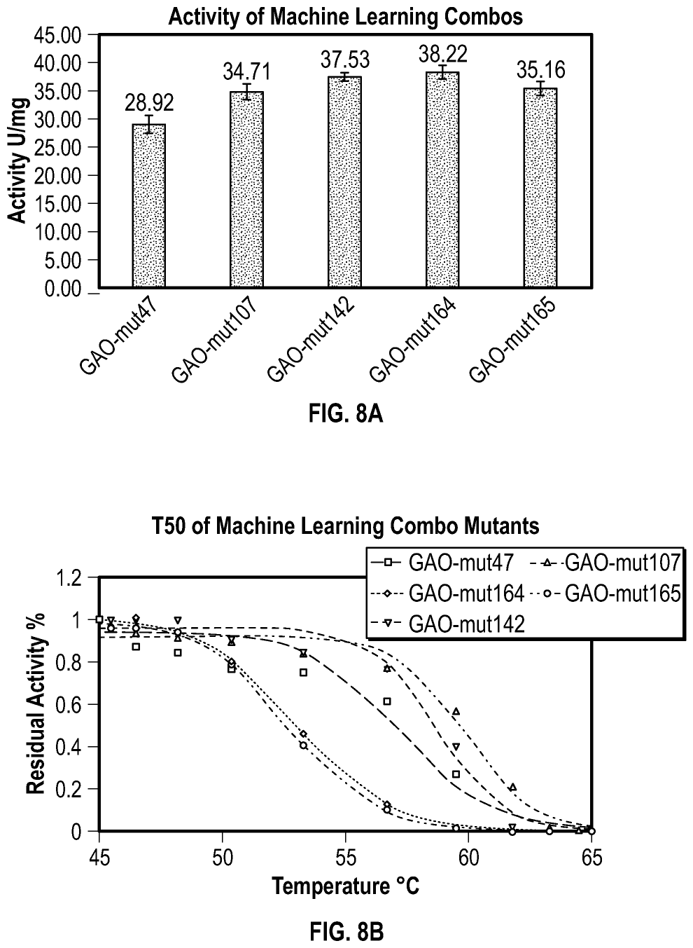

A one-step Parr Bomb Reaction was carried out with the GAO-Mut47 to produce D-glucodialdose. Specifically, a 50 ml reaction was conducted in a 200 mL vessel pressurized to 100 psi. The vessel was charged with 50 mM sodium phosphate pH 8 buffer, 50 μM CuSO₄, 15 w/v % glucose, 0.005 w/v % catalase, 0.001% horseradish peroxidase, and 0.001 w/v % engineered GAO. The reaction was stirred 500 rpm, 11° C. for 48 hours. Samples were taken at 0, 24, and 48 hours then assayed with HPLC to measure residual glucose and the results are shown in FIG. 7. The activity and stability of these GAO mutants containing mutations were further assayed by determining the specific activity rationally designed mutants compared with GAO-mut47 and GAO-Mut107 controls. These results are shown in FIG. 8A. A similar comparison of the T50 for these enzymes was made and the results are shown in FIG. 8B.

### Example 6

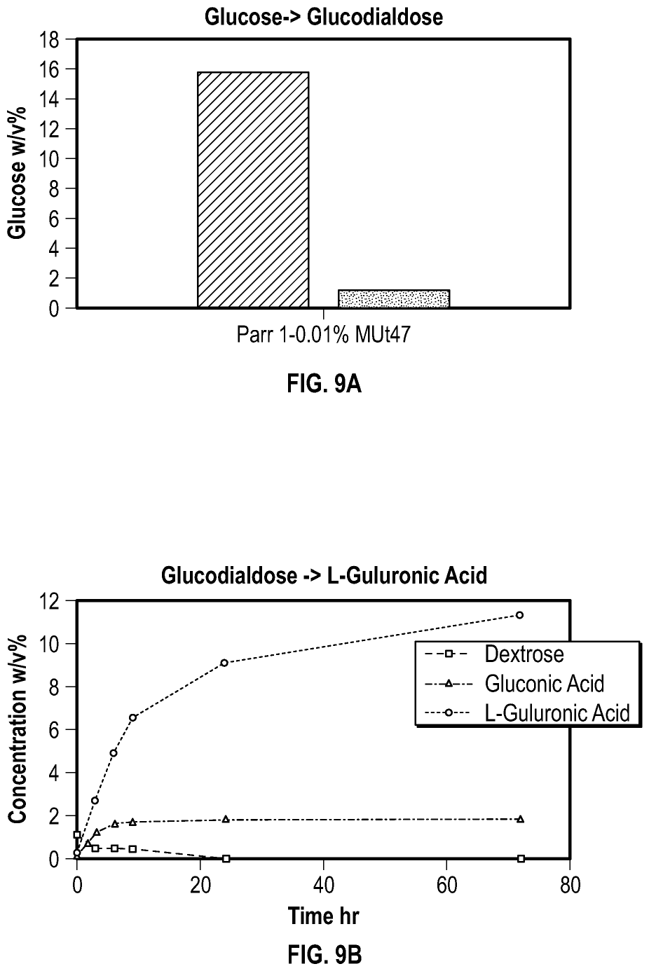

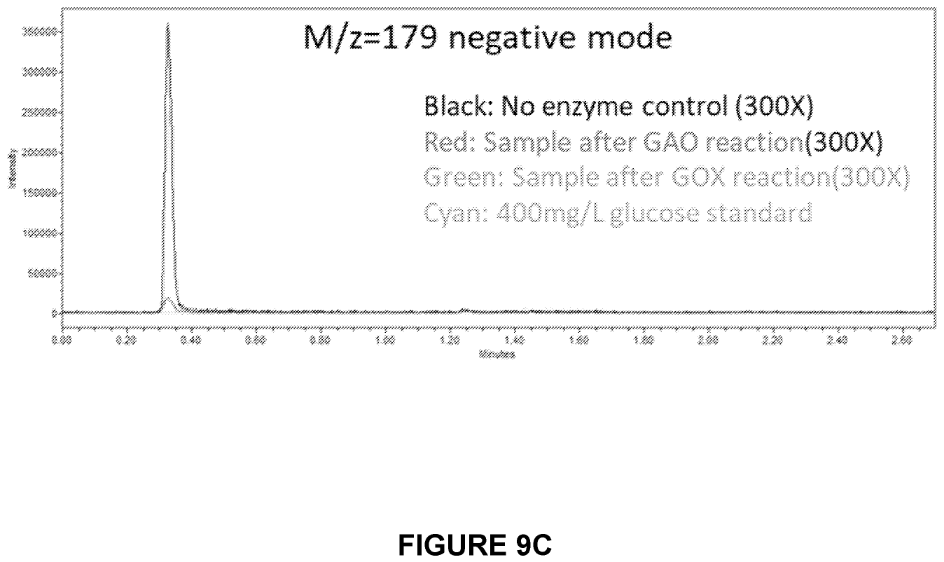

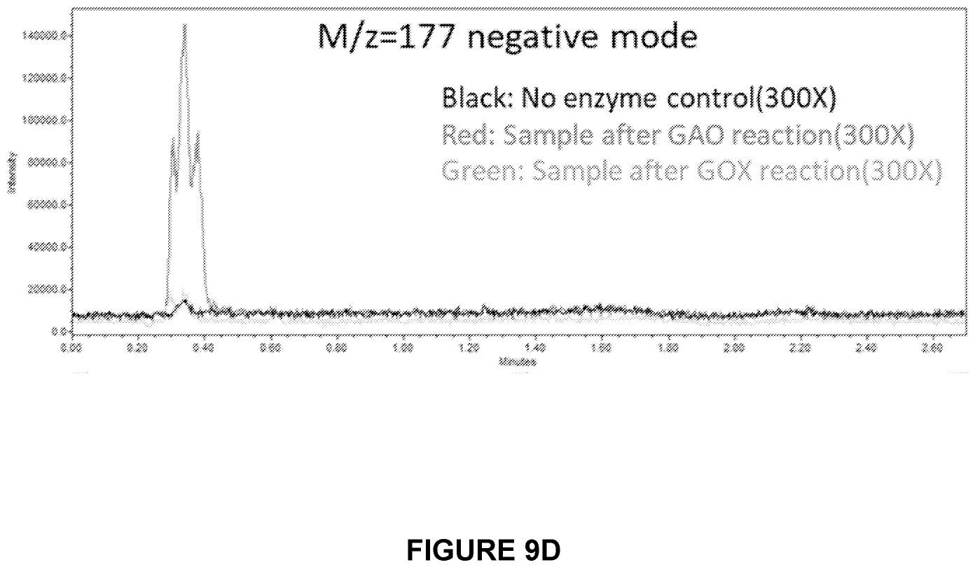

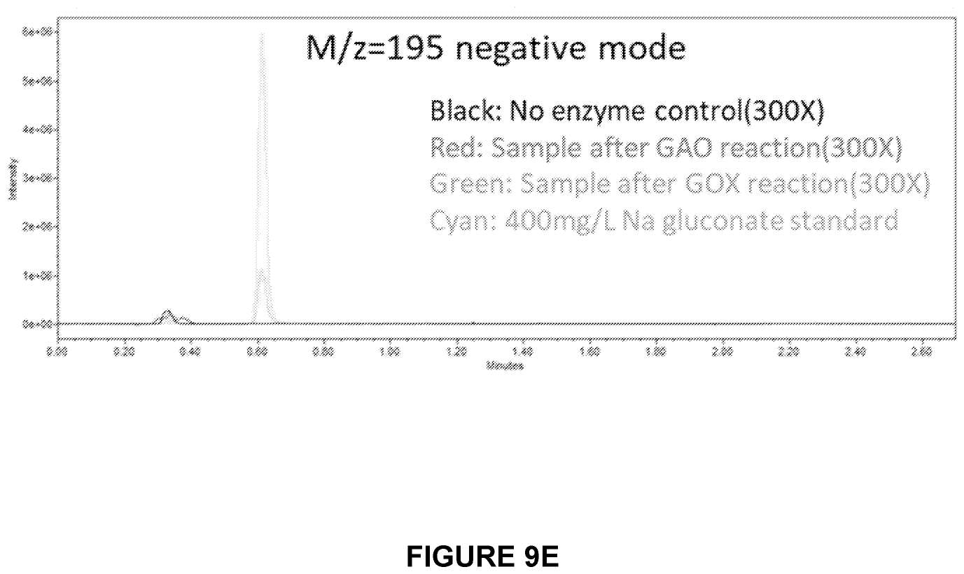

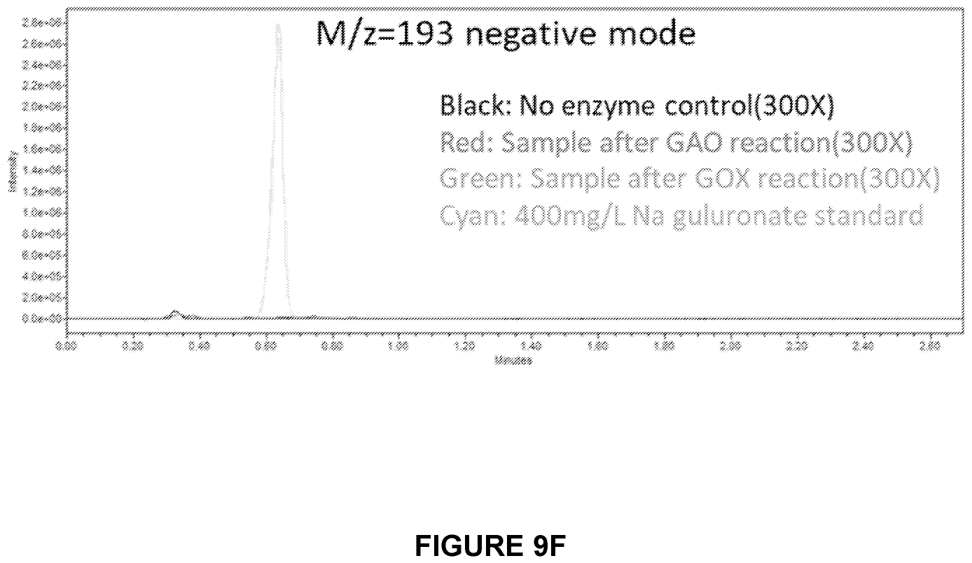

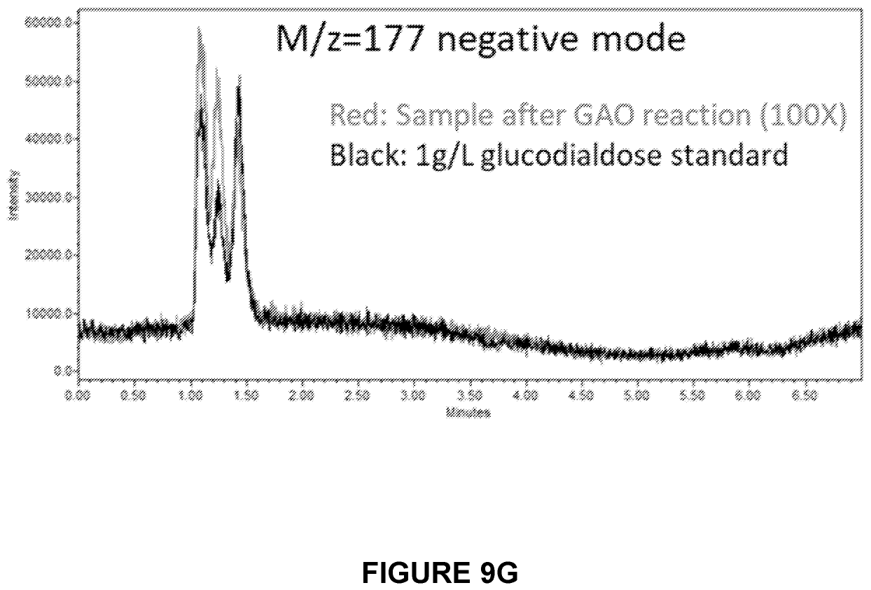

A two-step Parr Bomb Reaction with GAO-Mut47 was carried out to produce L-guluronic acid. A 50 ml reaction was conducted in a 200 mL vessel pressurized to 100 psi. The vessel was charged with 50 mM sodium phosphate pH 8 buffer, 50 μM CuSO₄, 15 w/v % glucose, 0.005 w/v % catalase, 0.001% horseradish peroxidase, and 0.01 w/v % engineered GAO. The reaction was stirred 500 rpm, 11° C. for 72 hours to generate glucodialdose from glucose. In the second step, 0.002% w/v GOX and an additional 0.001% w/v catalase was added and allowed to proceed at the same conditions for another 24 hours. The reaction was periodically paused and the pH adjusted to 7. Following the reaction with GAO-Mut47, the concentration of glucose dropped from the initial loading of 16% w/v to 1.5% w/v. After addition of GOX and reaction for 24 hours, a mixture of 2.0% w/v gluconic and 12% w/v L-guluronic acid (75% molar yield). These results are shown in FIG. 9.

### Example 7

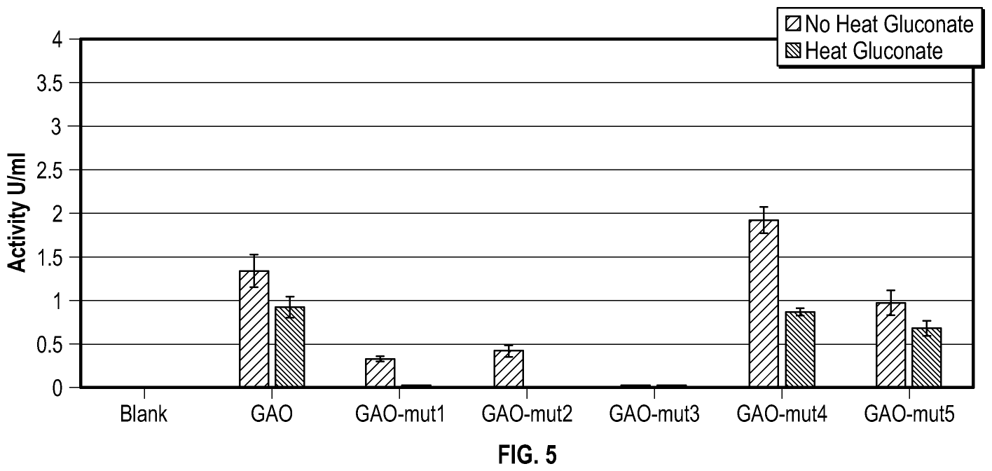

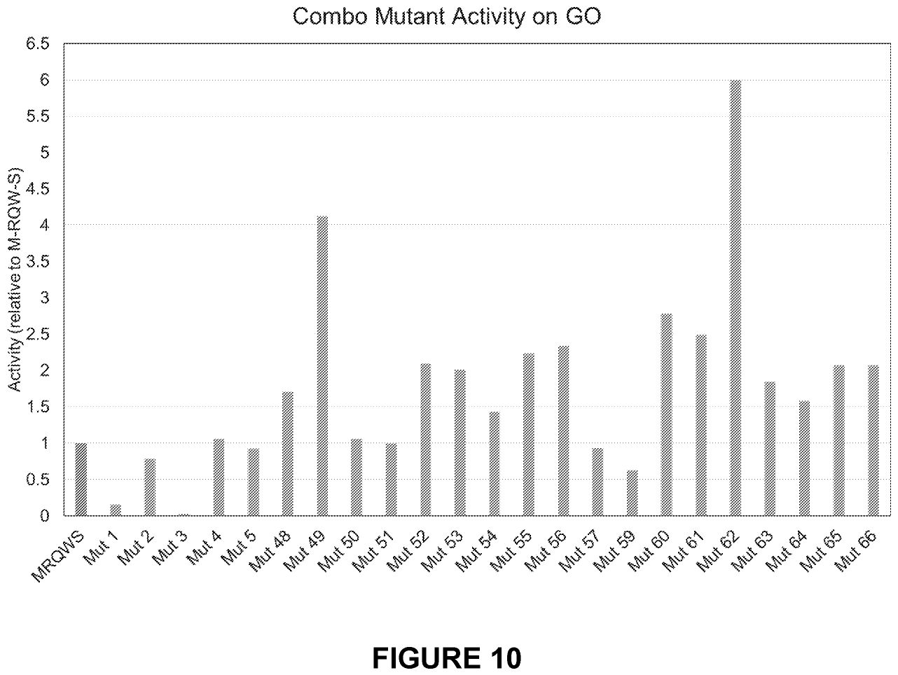

A GAO mutant for producing L-Guluronic Acid from gluconate or gluconolactone was produced. WT and GAO-Mut1-5 were probed for activity on gluconate and WT and GAO-mut4 were found to have a reasonable baseline level of activity (FIG. 5). As such, we probed combination mutants generated during the rational engineering of a GAO mutant active on glucose were screened for activity on gluconate. It was surmised that there may be mutants active on gluconate among the combinations generated based on a GAO-M-RQW-S background because the parent construct already demonstrated about 1 U mg⁻¹ specific activity on gluconate. Screening on purified protein with 2% gluconate revealed Mut49 (N66W, A172V, and Y189W) and Mut62 (N66S, A172V, Y189W, S306A, S311F, S331R, A178D, Q486L) as highly active on gluconate with specific activities of about 4 and 6 U mg⁻¹, respectively. The results are presented in FIG. 10.

### Example 8

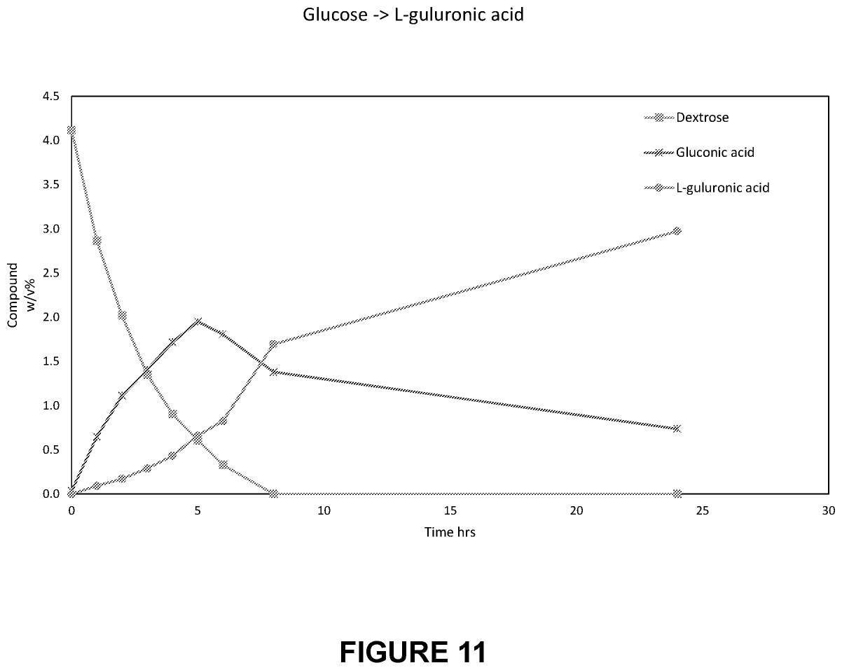

A one-step Parr Bomb reaction with GAO-Mut62 was carried out to produce L-guluronic acid. A 50 ml reaction was conducted in a 200 mL vessel pressurized to 100 psi. The vessel was charged with 50 mM sodium phosphate pH 8 buffer, 50 μM CuSO₄, 4 w/v % glucose, 0.005 w/v % catalase, 0.001% horseradish peroxidase, 0.0002 w/v % GOX, and 0.05 w/v % engineered GAO-mut62. The reaction was stirred 500 rpm, 11° C. for 24 hr. The reaction was periodically paused and the pH adjusted to 7.5. After 24 hours of reaction, 3 w/v % of L-guluronic acid and 1 w/v % of gluconic acid was generated from 4% glucose. The results are presented in FIG. 11.

While aspects of the presently disclosed subject matter have been shown and described, modifications thereof can be made by one skilled in the art without departing from the spirit and teachings of the subject matter. The aspects described herein are exemplary only, and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A chemoenzymatic process for the preparation of an oxidized glucose product comprising:
contacting D-glucose with an enzyme selected from the group consisting essentially of galactose oxidase (GAO), glucose oxidase (GOX), polysaccharide monooxygenase, catalase, animal peroxidase, periplasmic aldehyde oxidase (Pao), unspecific peroxygenase (UPO), lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), alpha-dioxygenase (aDox), dual oxidase (DuOx), prostaglandin H synthase (PGHS), cyclooxygenase (CyOx), linoleate diol synthase (LDS), variants thereof, and combinations thereof under conditions suitable for the formation of an oxidized intermediate; and
contacting the oxidized intermediate with a metal catalyst to form an oxidized glucose product.

2. The chemoenzymatic process of claim 1, wherein the galactose oxidase has SEQ ID NO.:1.

3. The chemoenzymatic process of claim 1, wherein the galactose oxidase has SEQ ID NO.:2.

4. The chemoenzymatic process of claim 1, wherein the galactose oxidase has any of SEQ ID NO.:6 to SEQ ID NO.:11.

5. The chemoenzymatic process of claim 1, wherein the glucose oxidase has SEQ ID NO.:3.

6. The chemoenzymatic process of claim 1, wherein the peroxidase is a lactoperoxidase.

7. The chemoenzymatic process of claim 6, wherein the lactoperoxidase has SEQ ID NO.:5.

8. The chemoenzymatic process of claim 1, wherein the polysaccharide monooxygenase has SEQ ID NO.:4.

9. The chemoenzymatic process of claim 1, carried out at a temperature of less than about 100° C.

10. The chemoenzymatic process of claim 1, wherein the oxidized glucose product has a purity of greater than about 80%.

11. The chemoenzymatic process of claim 1, wherein the oxidized glucose product comprises guluronic acid.

12. The chemoenzymatic process of claim 1, wherein the oxidized glucose product comprises glucaric acid.

13. The chemoenzymatic process of claim 1, wherein the metal catalyst comprises a support comprising carbon, silica, alumina, titania (TiO₂), zirconia (ZrO₂), zeolite, or any combination thereof.

14. The chemoenzymatic process of claim 1, wherein the metal catalyst is homogeneous.

15. The chemoenzymatic process of claim 1, wherein the metal catalyst is heterogeneous.

16. A chemoenzymatic process for the production of glucaric acid comprising:
contacting glucose with a galactose oxidase having any of SEQ ID NO.:6 to SEQ ID NO.:11. under conditions suitable for formation of D-glucohexodialdose;
contacting D-glucohexodialdose with a glucose oxidase having SEQ ID NO.:3 under conditions suitable for formation of L-guluronic acid-δ-2,6-lactone; and
contacting L-guluronic acid-δ-2,6-lactone with a heterogeneous metal catalyst under conditions suitable for the formation of glucaric acid.

17. A chemoenzymatic process for the production of D-glucono-δ-1,5-lactone comprising:
contacting glucose with a galactose oxidase having any of SEQ ID NO.:6 to SEQ ID NO.:11. and a glucose oxidase having SEQ ID NO.:3 under conditions suitable for the formation of D-glucono-δ-1,5-lactone.

18. A chemoenzymatic process for the production of glucaric acid comprising:
acidifying D-glucono-δ-1,5-lactone to form L-gluconate;
contacting L-gluconate with a galactose oxidase having any of SEQ ID NO.:6 to SEQ ID NO.:11. and a glucose oxidase having SEQ ID NO.:3 under conditions suitable for the formation of L-guluronate: and
contacting L-guluronate with a heterogeneous metal catalyst to form glucaric acid.

19. A chemoenzymatic process for the production of glucaric acid comprising:
contacting a polysaccharide monooxygenase having SEQ ID NO.:4. under conditions suitable for formation of saccharic acid lactone; and
hydrolyzing saccharic acid lactone at a pH of greater than about 7 to form glucaric acid.

20. A chemoenzymatic process for the production of glucaric acid comprising:
contacting glucose with an enzyme composition comprising a glucose oxidase having SEQ ID NO.:3, a peroxidase, halide ions, and a nitroxyl radical mediator, under conditions suitable for the formation to form an oxidized glucose intermediate; and contacting the oxidized glucose intermediate with a heterogeneous catalyst under conditions suitable for the formation of glucaric acid.

21. The chemoenzymatic process of claim 20, wherein the nitroxyl radical mediator comprises 2,2,6,6-tetramethylpiperidine N-oxyl (TEMPO) phthalimide N-oxyl or a combination thereof.

22. A manufacturing process comprising:
introducing to a reactor a feedstock comprising glucose and an enzyme selected from the group consisting essentially of galactose oxidase (GAO), glucose oxidase (GOX), polysaccharide monooxygenase, catalase, animal peroxidase, periplasmic aldehyde oxidase (Pao), unspecific peroxygenase (UPO), lactoperoxidase (LPO), myeloperoxidase (MPO), eosinophil peroxidase (EPO), thyroid peroxidase (TPO), ovoperoxidase, salivary peroxidase, vanadium haloperoxidase, non-mammalian vertebrate peroxidase (POX), peroxidasin (Pxd), bacterial peroxicin (Pxc), invertebrate peroxinectin (Pxt), short peroxidockerin (PxDo), short peroxidockerin (Pxt), alpha-dioxygenase (aDox), dual oxidase (DuOx), prostaglandin H synthase, cyclooxygenase (PGHS/CyOx), linoleate diol synthase (LDS), variants thereof and combinations thereof;
operating the reactor under conditions suitable for the formation of a feedstock comprising an oxidized glucose with an aldehyde moiety;
transferring the feedstock comprising an oxidized glucose with an aldehyde moiety to another reactor comprising a heterogeneous metal catalyst; and
operating the another reactor under conditions suitable for the oxidation of the feedstock.
